(19) United States
(12) Patent Application Publication
(10) Pub. No.: US 2009/0258850 A1
(43) Pub. Date: Oct. 15, 2009

(54) STABILIZED THERAPEUTIC COMPOSITIONS AND FORMULATIONS

(76) Inventors: James M. Frincke, San Diego, CA (US); Steven K. White, San Diego, CA (US); Yu Ge, San Diego, CA (US); Weicheng Liaw, San Diego, CA (US); Tao Hu, San Diego, CA (US)

Correspondence Address: HOLLIS-EDEN PHARMACEUTICALS, INC., 4435 EASTGATE MALL, SUITE 400, SAN DIEGO, CA, 92121, US

(21) Appl. No.: 12/196,271

(22) Filed: Aug. 21, 2008

Related U.S. Application Data
(60) Provisional application No. 60/965,730, filed on Aug. 21, 2007.

(51) Int. Cl. A61K 31/5685

(52) U.S. Cl. 514/182

(57) ABSTRACT

The invention relates to pharmaceutically acceptable formulations comprising an active pharmaceutical ingredient such as androst-5-ene-3β,17β-diol, androst-5-ene-3β,7β,17β-triol or derivatives of either of these compounds and an air oxidizable excipient that have been stabilized with respect to efficacy. Use of the efficacy-stabilized formulations to treat a number of conditions or symptoms thereof, such as a symptom associated with exposure to radiation is described.

### CROSS-REFERENCE TO RELATED APPLICATIONS

This application claims priority to pending U.S. provisional application Ser. No. 60/965,730 filed Aug. 21, 2007, which is incorporated herein by reference.

### FIELD OF THE INVENTION

The invention relates to compositions and formulations for parenteral or other routes of administration that are stabilized with regard to their biological activity or efficacy and the use of these compositions and formulations for treating conditions related to immune responses, blood disorders and radiation exposure.

### BACKGROUND OF THE INVENTION

Parenteral administration is a route of administration other than by way of the digestive tract. A therapeutic pharmaceutical is typically administered in a parenteral dosage form when the pharmaceutical has insufficient oral bioavailability to elicit the desired therapeutic effect. For some formulations, e.g., where the active pharmaceutical has poor water solubility, an aqueous-based composition or formulation can be an emulsion or suspension dosage form that is intended for administration by a route other than intravenous injection, such as intramuscular, intradermal or subcutaneous injection.

In some parenteral dosage forms, a surface-active agent (surfactant) can sometimes be used to provide an acceptable injection volume to deliver sufficient active pharmaceutical ingredient in a therapeutically effective amount of a pharmaceutically acceptable composition or formulation. Flocculated suspensions have received some attention due to their favorable resuspendability characteristics, although maintaining their stability can be problematic. Controlled flocculation of particles can sometimes be obtained by the use of flocculating agents that provides a zeta potential surrounding the solid particles, which allow for formation of a loose aggregate of particles that are microscopically separated from each other and are readily re-suspendable. Substances that initiate flocculation can include ionic or amphoteric surfactants or a combination of thereof that can form a bridge between particles.

Pharmaceutical formulations typically are characterized by an acceptable range of parameters, such as pH and/or a relative proportion of the drug or active pharmaceutical ingredient (API) to isomers of the API. These parameters can sometimes change on storage of a formulation. New chemical species or degradants in a formulation can potentially arise from a number of sources, e.g., from an inherent instability of the API due to epimerization of an atom or chemical group, or alteration of one or more excipients. Such degradants can be relatively benign, with limited effect on the shelf life or biological efficacy of the formulation or they can adversely affect the shelf life or efficacy of the formulation. This depends to a large extent on the acceptable parameter range and the rate at which those parameters may change. Epimerization or loss of one or more atoms or groups of an API can lead to a reduced shelf life or biological efficacy. Alteration of one or more excipients in formulations can also occur over time. Thus, oxidation or isomerization of an excipient or the API can sometimes adversely alter these components in an otherwise pharmaceutically acceptable composition or formulation.

Polysorbate 80 has been used, e.g., as a surface-active agent in some suspension dosage forms or formulations. Auto-oxidation of Polysorbate 80 is described in Donbrow, M, et al., J. Pharm. Sci. 1978, 67:1676-1681 and Hamburger, E, et al. Pharm. Acta Helv. 1975, 50:10-17. Formation of auto-oxidation degradants may have no discernable effects on a given formulation or they may be associated with an unwanted chemical modification of the active pharmaceutical ingredient, a decrease in pH or a decrease in suspendability of excipients and/or API in some suspension formulations. Physiochemical stability characteristics of some suspension dosage forms containing Polysorbate 80 are described in MacLeod, et al. US Pat. Appl. No. 2003/0114430, Columbo, et al., US Pat. Appl. No. 2003/0130245 and Gao, et al. PCT Publication No. WO 02/102376.

Most pharmaceutical formulations such as parenteral formulations will have an acceptable potency or efficacy range for the API and an acceptable range of parameters associated with excipients or the formulation itself, e.g., pH or metal ion content. For example, in parenteral formulations that contain a relatively potent drug or API with a relatively small effective dose for a human, e.g., about 100 μg to 300 mg, a relatively broad range of formulation parameters such as relative API purity, pH or heavy metal ion concentration (expressed as lead equivalents) may be acceptable because only a small volume (less than, e.g. 4 mL or more typically 2 mL or less) of the formulation may deliver the needed API dose. Because of such considerations, it is simply not predictable in advance if an API or excipient(s) in any given formulation will be characterized by a relatively rapid or a relatively slow change such that the shelf life and/or biological efficacy of the formulation is significantly affected. When efficacy or shelf life of a parenteral or other formulation is found to be adversely affected, there are many potential means to consider as ways to potentially improve or stabilize the formulation. Such avenues include decreasing the dosage by altering the route of administration, e.g., from oral administration to parenteral administration, increasing the acceptable pH range for the. formulation, increasing the relative potency of the API by using a more purified preparation, using a different physical form of API and/or using other options.

### SUMMARY OF THE INVENTION

It has been surprisingly found that certain parenteral suspension formulations containing a compound such as androst-5-ene-3β,17β-diol as the active pharmaceutical ingredient (API), may have limited or no efficacy after storage of the invention composition or formulation. This change in activity or efficacy can arise despite acceptable or no observed changes over time in parameters including the strength or relative purity of the API and the suspendability of API and/or excipients in the suspension formulations. By use of the invention compositions, formulations and methods disclosed herein, it has been found that the biological efficacy of the invention compositions and formulations is retained on storage of a parenteral dosage form that contains an air oxidizable excipient. The presence of the air oxidizable excipient was found, in some formulations, to be associated with a decrease of biological efficacy of the suspension formulation such that the useful shelf life of the formulation was greatly reduced. This loss of parenteral formulation efficacy was not associated with a significant change in pH (e.g., a 4 pH unit decrease), which was particularly unexpected with an injection dependent route of administration when no other adverse physiochemical changes (e.g., loss of API strength) were observed, since parenteral administration of an invention composition or formulation so effected would result in rapid equalization to physiological pH at the injection site. Without being bound by theory, it is believed that in the invention compositions and formulations described herein, a degradant(s) arises from an air oxidizable excipient(s), potentially from an air oxidizable surface-active agent, in certain suspension dosage forms or formulations. This degradant(s) may induce the observed decreased efficacy or limited shelf life of certain dosage forms or suspension formulations either as a direct consequence of the presence of the degradant(s) or as an event associated with its formation.

The invention compositions, formulations or methods accomplish one or more of the following objects. One object is to provide pharmaceutically acceptable, efficacy stabilized formulations and invention compositions for parenteral administration to a subject wherein the formulation or invention composition comprises an active pharmaceutical ingredient and at least one air oxidizable excipient. In general, the invention formulations are sterile aqueous suspension formulations intended for oral, buccal, sublingual or, more often, parenteral administration, e.g., subcutaneous, intradermal or, more typically, intramuscular injection.

Another object is to provide efficacy stabilized compositions or formulations wherein the active pharmaceutical ingredient is a formula 1 compound (F1C) wherein the F1C is androst-5-ene-3β,17β-diol, androst-5-ene-3β,7β,17β-triol or a mono-, di- or tri-ester or ether derivative of either of these compounds. Especially preferred are formulations comprising the compound androst-5-ene-3β,17β-diol. Such preferred formulations usually are suspension formulations suitable for subcutaneous, intradermal or, more typically, intramuscular injection.

Another object is to provide methods to prepare sterilized pharmaceutically acceptable invention compositions or formulations that are efficacy stabilized.

Another object is to provide an article of manufacture comprising an active pharmaceutical ingredient, such as a F1C, in an efficacy stabilized parenteral dosage form in a container system wherein an oxygen-depleted internal atmosphere is maintained.

Invention objects also include formula 1 compounds as efficacy stabilized invention compositions or formulations for parenteral administration that are useful to treat or ameliorate one or more symptoms of a pathological condition associated with immune suppression, deficient Th1 immune responses, an unwanted immune response, a blood disorder or radiation exposure.

Other invention objects include methods of treating one or more symptoms of a pathological condition associated with immune suppression, deficient Th1 immune responses, an unwanted immune response, a blood disorder or radiation exposure with parenteral dosage forms of an active pharmaceutical ingredient such as a formula 1 compound.

Another invention object provides a method of providing to a patient in need thereof a poorly water soluble or water insoluble active pharmaceutical ingredient, such as a F1C having pharmacological activity in an efficacy stabilized suspension dosage form by intramuscular or subcutaneous administration of the suspension.

Another invention object is an efficacy stabilized suspension comprising a formula 1 compound, such as androst-5-ene-3β,17β-diol for parenteral administration to a subject having one or more symptoms from radiation exposure.

### BRIEF DESCRIPTION OF THE DRAWINGS

FIG. 1 provides peroxide levels in an androst-5-ene-3β,17β-diol formulation with one excipient omitted with Polysorbate 80 as the air oxidizable excipient having PV of 2 mequiv O₂/Kg.

FIG. 2 provides formaldehyde levels in an androst-5-ene-3β,17β-diol formulation or formaldehyde levels with one excipient omitted with Polysorbate 80 as the air oxidizable excipient having PV of 2 mequiv O₂/Kg.

FIG. 3 provides the effect of pre-treatment of an androst-5-ene-3β,17β-diol formulation on formaldehyde levels with Polysorbate 80 as the air oxidizable excipient having PV of 2 mequiv O₂/Kg.

FIG. 4 provides a CMF plot for platelet effects obtained with a formulation comprising androst-5-ene-3β,17β-diol and Polysorbate 80 as the air oxidizable excipient.

FIG. 5 provides the therapeutic effect of a stabilized formulation comprising androst-5-ene-3β,17β-diol and Polysorbate 80 as the air oxidizable excipient (Solid line: Stabilized AED Formulation; Broken Line: Vehicle)

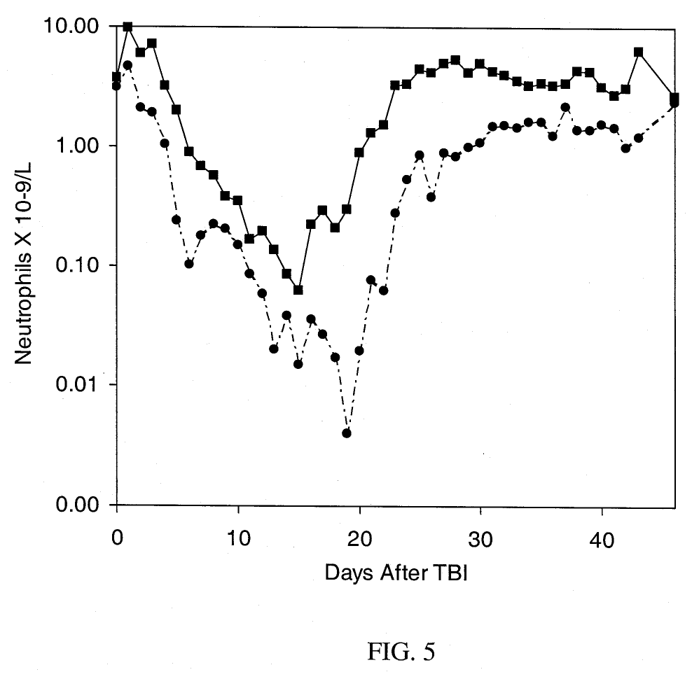

FIG. 6 provides the loss of therapeutic effect of a formulation comprising androst-5-ene-3β,17β-diol and Polysorbate 80 as the air oxidizable excipient without stabilization (Solid line: Non-Stabilized AED Formulation; Broken Line: Vehicle)

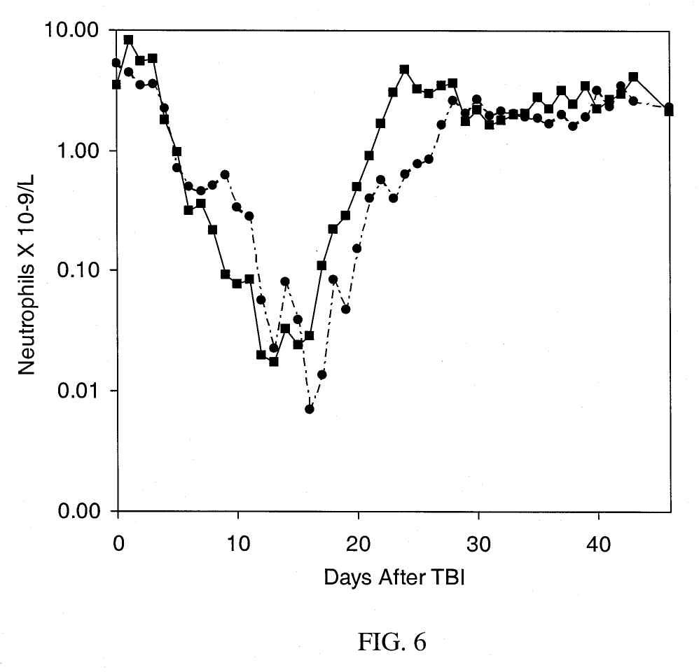

### DETAILED DESCRIPTION

### Definitions

As used herein and unless otherwise stated or implied by context, terms that are used herein have the meanings defined below. Unless otherwise contraindicated or implied, e.g., by including mutually exclusive elements or options, in these definitions and throughout this specification, the terms “a” and “an” mean one or more and the term “or” means and/or.

Position numbers that are given for compounds of Formula 1 (F1Cs) use the numbering convention for cholesterol.

A “subject” means a human or animal. Usually the animal is a mammal or vertebrate such as a human or a non-human primate, rodent, lagomorph, domestic animal or game animal. Non-human primates include chimpanzees, Cynomolgus monkeys, spider monkeys, and macaques, e.g., Rhesus or Pan. Rodents and lagomorphs include mice, rats, woodchucks, ferrets, rabbits and hamsters. Domestic and game animals include cows, horses, pigs, sheep, deer, bison, buffalo, mink, felines, e.g., domestic cat, canines, e.g., dog, wolf and fox, avian species, e.g., chicken, turkey, emu and ostrich, and fish, e.g., trout, catfish and salmon. Typically, a subject will be a human, a non-human primate, a dog or a rodent (e.g., a mouse or rat).

At various locations in the present disclosure, e.g., in any disclosed embodiments or in the claims, reference is made to compounds, compositions, or methods that “comprise” one or more specified components, elements or steps. Invention embodiments also specifically include those compounds, compositions, compositions or methods that are or that consist of or that consist essentially of those specified components, elements or steps. For example, disclosed compositions or methods that “comprise” a component or step are open and they include or read on those compositions or methods plus an additional component(s) or step(s). Similarly, disclosed compositions or methods that “consist of” a component or step are closed and they would not include or read on those compositions or methods having appreciable amounts of an additional component(s) or an additional step(s).

“Alkyl” as used here means linked normal, secondary, tertiary or cyclic carbon atoms, i.e., linear, branched, cyclic or any combination thereof. Alkyl moieties, as used herein, may be saturated, or unsaturated, i.e., the moiety may comprise one, two, three or more independently selected double bonds or triple bonds. Unsaturated alkyl moieties include moieties as described below for alkenyl, alkynyl, cycloalkyl, and aryl moieties. Saturated alkyl groups contain saturated carbon atoms (sp³) and no aromatic, sp² or sp carbon atoms. The number of carbon atoms in an alkyl group or moiety can vary and typically is 1 to about 50, e.g., about 1-30 or about 1-20, more typically and preferred is 1-8 or 1-6 carbon atoms. Unless otherwise specified, e.g., C₁₋₈ alkyl or C1-C8 alkyl means an alkyl moiety containing 1, 2, 3, 4, 5, 6, 7 or 8 carbon atoms and C₁₋₆ alkyl or C1-C6 means an alkyl moiety containing 1, 2, 3, 4, 5 or 6 carbon atoms. When an alkyl group is specified, species may include, by way of example and not limitation, methyl, ethyl, 1-propyl (n-propyl), 2-propyl (iso-propyl, —CH(CH₃)₂), 1-butyl (n-butyl), 2-methyl-1-propyl (iso-butyl, —CH₂CH(CH₃)₂), 2-butyl (sec-butyl, —CH(CH₃)CH₂CH₃), 2-methyl-2-propyl (t-butyl, —C(CH₃)₃), amyl, isoamyl, sec-amyl, 1-pentyl (n-pentyl), 2-pentyl (—CH(CH₃)CH₂CH₂CH₃), 3-pentyl (—CH(CH₂CH₃)₂), 1 -hexyl, 2-hexyl (—CH(CH₃)CH₂CH₂CH₂CH₃), 3-hexyl (—CH(CH₂CH₃)(CH₂CH₂CH₃)), cyclopropyl, cyclobutyl, cyclopentyl, cyclohexyl and cycloheptyl.

Cycloalkyl as used here is a monocyclic, bicyclic or tricyclic ring system composed of only carbon atoms. The number of carbon atoms in a cycloalkyl group or moiety can vary and typically is 3 to about 50, e.g., about 3-30 or about 3-20, more typically and preferred is 3-8 or 3-6 carbon atoms. Unless otherwise specified, e.g., C₃₋₈ alkyl or C3-C8 alkyl means a cycloalkyl moiety containing 3, 4, 5, 6, 7 or 8 carbon atoms and C₃₋₆ alkyl or C3-C6 means a cycloalkyl moiety containing 3, 4, 5 or 6 carbon atoms. When a cycloalkyl group is specified, species may include cyclopropyl, cyclopentyl, cycohexyl, cycloheptyl and adamantly.

“Alkenyl” as used here means a moiety that comprises one or more double bonds (—CH═CH—), e.g., 1, 2, 3, 4, 5, 6 or more, typically 1, 2 or 3 and can include an aryl moiety such as benzene, and additionally comprises linked normal, secondary, tertiary or cyclic carbon atoms, i.e., linear, branched, cyclic or any combination thereof unless the alkenyl moiety is vinyl (—CH═CH₂). An alkenyl moiety with multiple double bonds may have the double bonds arranged contiguously (i.e. a 1,3 butadienyl moiety) or non-contiguously with one or more intervening saturated carbon atoms or a combination thereof, provided that a cyclic, contiguous arrangement of double bonds do not form a cyclically conjugated system of 4n+2 electrons (i.e., aromatic). The number of carbon atoms in an alkenyl group or moiety can vary and typically is 2 to about 50, e.g., about 2-30 or about 2-20, or, preferably 2-6 or 2-8, unless otherwise specified, e.g., C₂₋₈ alkenyl or C2-8 alkenyl means an alkenyl moiety containing 2, 3, 4, 5, 6, 7 or 8 carbon atoms and C₂₋₆ alkenyl or C2-6 alkenyl means an alkenyl moiety containing 2, 3, 4, 5 or 6 carbon atoms. Alkenyl groups will typically have 2, 3, 4, 5, 6, 7, 8, 9, 10, 11, 12, 13, 14, 15, 16, 17, 18, 19 or 20 carbon atoms. When an alkenyl group is specified, species include, e.g., any of the alkyl moieties described above that has one or more double bonds, methylene (═CH₂), methylmethylene (═CH—CH₃), ethylmethylene (═CH—CH₂—CH₃), ═CH—CH₂—CH₂—CH₃, vinyl (—CH═CH₂), allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, —(CH₂)ₙ—(CH═CH)—(CH₂)ₘ—CH₃, —(CH₂)ₙ—(CCH₃═CH)—(CH₂)ₘ—CH₃, —(CH₂)ₙ—(CH═CCH₃)—(CH₂)ₘ—CH₃, —(CH₂)ₙ—(CH═CH)₀₋₁—(CH₂)ₘ—CH₂CH═CH₂ and —(CH₂)ₙ—(CH═CH)₀₋₁—(CH₂)ₘ—CH₂—(CH═CH)₀₋₁—CH₃, where n and m independently are 0, 1, 2, 3, 4, 5, 6, 7 or 8.

“Alkynyl” as used here means a moiety that comprises one or more triple bonds (—C≡C—), e.g., 1, 2, 3, 4, 5, 6 or more, typically 1 or 2 triple bonds, optionally comprising 1, 2, 3, 4, 5, 6 or more triple bonds, with the remaining bonds (if present) being single bonds and comprising linked normal, secondary, tertiary or cyclic carbon atoms, i.e., linear, branched, cyclic or any combination thereof, unless the alkynyl moiety is ethynyl. The number of carbon atoms in an alkynyl group or moiety can vary and typically is 2 to about 50, e.g., about 2-30 or about 2-20 or preferably 2-6. Unless otherwise specified, e.g., C₂₋₆ alkynyl or C2-6 alkynyl means an alkynyl moiety containing 2, 3, 4, 5, 6, 7 or 8 carbon atoms. When an alkynyl group is specified, species include, e.g., any of the alkyl moieties described above that has one or more triple bonds, butynyl, iso-butynyl, —CCH, —CCCH₃, —CCCH₂CH₃, —CCC₃H₇, —CCCH₂C₃H₇, —(CH₂)ₙ—(C≡C)—(CH₂)ₘ—CH₃, —(CH₂)ₙ—(C≡C)₀₋₁—(CH₂)ₘ—CH₂C≡CH, —(CH₂)ₙ—(C≡C)₀₋₁—(CH₂)ₘ—CH₂—(C≡C)₀₋₁—CH₃, —(CH₂)ₙ—(C≡C)—CH₂—(C≡C)—(CH₂)ₘ—CH₃, where each n and m independently are 0, 1, 2, 3, 4, 5, 6, 7 or 8.

“Aryl” as used here means an aromatic ring system or a fused ring system with no ring heteroatoms comprising 1,2, 3 or 4 to 6 rings, typically 1 to 3 rings; wherein the rings are composed of only carbon atoms; and refers to a cyclically conjugated system of 4n+2 electrons (Hückel rule), typically 6, 10 or 14 electrons some of which may additionally participate in exocyclic conjugation (cross-conjugated). When an aryl group is specified, species may include phenyl, naphthyl, phenanthryl and quinone.

“Substituted alkyl”, “substituted cycloalkyl”, “substituted alkenyl”, “substituted alkynyl”, substituted alkylaryl”, “substituted arylalkyl”, “substituted heterocycle”, “substituted aryl”, and the like mean an alkyl, alkenyl, alkynyl, aryl or other group or moiety as defined or disclosed herein that has a substituent(s) that replaces a hydrogen atom(s) or a substituent(s) that interrupts a carbon atom chain. Substituted heterocycles may thus have a substituent bonded to a ring carbon or a ring heteroatom such as nitrogen.

“Optionally substituted alkyl”, “optionally substituted alkenyl”, “optionally substituted alkynyl”, “optionally substituted alkylaryl”, “optionally substituted arylalkyl”, “optionally substituted heterocycle”, “optionally substituted aryl”, “optionally substituted heteroaryl”, “optionally substituted alkylheteroaryl”, “optionally substituted heteroarylalkyl”, “optionally substituted monosaccharide” and the like mean an alkyl, alkenyl, alkynyl, alkylaryl, arylalkyl heterocycle, aryl, heteroaryl, alkylheteroaryl, heteroarylalkyl, monosaccharide or other group or moiety as defined or disclosed herein that has a substituent(s) that optionally replaces a hydrogen atom(s) or a substituent(s) that interrupts a carbon atom chain. Such substituents are as described above. For a phenyl moiety, the arrangement of any two substituents present on the aromatic ring can be ortho (o), meta (m), or para (p) relative to each other.

“Ester” as used here means a moiety that contains a —C(O)—O— structure. Typically, esters as used here comprise an organic moiety containing about 1-50 carbon atoms (e.g., about 2-20 carbon atoms) and 0 to about 10 independently selected heteroatoms (e.g., O, S, N, P, Si), typically 0-2 heteroatoms, where the organic moiety is bonded to a formula 1 steroid nucleus at, e.g., a hydroxyl moiety through the —C(O)—O— structure, e.g., organic moiety-C(O)—O-steroid organic moiety-O—C(O)-steroid. The organic moiety usually comprises one or more of any of the organic groups described herein, e.g., C₁₋₆ alkyl moieties, C₂₋₆ alkenyl moieties, C₂₋₆ alkynyl moieties, aryl moieties, C₂₋₉ heterocycles or substituted derivatives of any of these, e.g., comprising 1, 2, 3, 4 or more substituents, where each substituent is independently chosen. Exemplary substitutions for hydrogen or carbon atoms in these organic groups are as described above for substituted alkyl and other substituted moieties. Substitutions are independently chosen. Exemplary esters are typically hydroxyl esters and include by way of example and not limitation, one or more independently selected acetate, propionate, isopropionate, isobutyrate, butyrate, valerate, caproate, isocaproate, hexanoate, heptanoate, octanoate, nonanoate, decanoate, undecanoate, phenylacetate or benzoate esters. Preferred esters are acetate and propionate with acetate particularly preferred.

“Ether” as used here means an organic moiety as described for ester that comprises 1, 2, 3, 4 or more —O— moieties, usually 1 or 2. In some embodiments, the —O— group is linked to the steroid nucleus at a hydroxy moiety. Preferred ethers are C1-6 ethers with methoxy and ethoxy particularly preferred.

An “invention formulation” or “formulation” as used herein is a composition, comprising a blend of at least one F1C or hydrate thereof, usually 1 or 2, or at least one pharmaceutically acceptable salt of a F1C and one or more excipients, typically two, three or more excipients. In general, formulations will be suspensions that are administered parenterally to a subject without further manipulations that change the ingredients or the ingredient proportions that are present immediately prior to the manipulation.

An “invention composition” is a composition that is an intermediate one can use to make the invention formulations, i.e., a change(s) in an ingredient(s) or its amount(s) is needed to make a formulation. Thus, invention compositions include compositions where further processing is required before it is a formulation, e.g., mixing or addition of a desired amount of an ingredient such as a diluent (e.g. vehicle).

“Parenteral administration” as used here means introduction of a pharmacologically active compound, composition or formulation to a subject through a route other than the digestive system and includes injection dependent routes such as intravenous, subcutaneous, intradermal, epidural, intraperitoneal, intramuscular, intramedullary, intraorbital, intracapsular, intraspinal, intrathecal or intrasternal and injection independent routes such as topical, intranasal, ophthalmic or inhalation.

“Pharmaceutically acceptable” as used herein in reference to the different composition or formulation components, or the composition or formulation itself, means that the components of the composition or formulation itself do not cause unacceptable adverse side effects in relation to the condition and the subject being treated. Examples of pharmaceutically acceptable components are provided in United States Pharmacopoeia and National Formulary, USP 30-NF 25, May 2007 (hereby specifically incorporated by reference herein into the present application).

“Efficacy stabilized formulation” or “efficacy stabilized composition” as used herein means a invention composition or formulation wherein one of more degradants derivable from a pharmaceutically acceptable excipient have been removed either prior to or after blending to provide a composition or formulation such that the composition or formulation so treated retains a significant fraction of its efficacy for its intended purpose after exposure to ambient or other storage temperatures. A composition or formulation may be further stabilized by addition of another pharmaceutically acceptable excipient that inhibits further formation of the degradant(s).

“Parenteral composition” or “parenteral formulation” as used here means an invention composition or formulation suitable for parenteral administration of an active pharmaceutical ingredient such as a F1C. Pharmaceutically acceptable invention compositions or formulations suitable for parenteral administration in human or veterinary applications include, by way of example and not limitation, liquid solutions, suspensions, emulsions, gels, creams, intramammary infusions, intravaginal delivery systems and implants.

An “excipient” as used herein means a component or an ingredient, other than the active pharmaceutical ingredient, that is included in a invention composition or formulation and has been found acceptable in the sense of being compatible with the other ingredients of invention compositions or formulations and has been appropriately evaluated for safety and found not overly deleterious to the patient or animal to which the invention composition or invention formulation is to be administered. Excipients typically used in the pharmaceutical formulation arts include diluents, disintegrants, binders, anti-adherents, lubricants, glidants, sorbents, suspension agents, dispersion agents, wetting agents, surface-active agents, flocculating agents, buffering agents, tonicity-adjusting agents, metal chelator agents, anti-oxidants, preservatives, fillers, flow enhancers, compression aids, colors, sweeteners, film formers, film coatings, favors and printing inks. Examples of excipients, by way of illustration and not limitation, used in the preparation of an invention composition or formulation are given in Nema, S., et al. PDA J. Pharm. Sci. Tech. 1997, 51:166-171; Strickley, R. G. Pharm. Res. 2004, 21:201-230; Powell, M. F., et al. PDA J. Pharm. Sci. Tech 1998, 52:238-311; Akers, M. J. in “Drug Delivery: Parenteral Route” Encyclopedia of Pharmaceutical Technology, Informa Healthcare, USA, 2007, pp 1266-1278 (hereby specifically incorporated by reference into the present application).

A “suspension” as used here unless specified or implied by context is a F1C that is usually suspended as a finely divided solid in a liquid carrier (vehicle) at a time before administration. The suspension may be either ready to use or a dry powder reconstituted as a suspension dosage form just prior to use (e.g., by adding water for injection or a buffered aqueous solution. Suspensions are used when an active pharmaceutical ingredient such as a F1C compound is insoluble or poorly soluble in a desired diluent or vehicle and typically include a suspending or flocculating agent, a wetting agent, if the suspending or flocculating agent that is present does not already serve this purpose, a buffering agent and a preservative. In a colloidal suspension, the F1C particles are less than about 1 μm in size. In a coarse suspension, they are larger than about 1 μm (e.g. about 2-20 μm ). The practical upper limit for individual suspendable F1C particles in coarse suspensions is about 50 μm to 75 μm although particles up to 200 μm may be suitable. Parenteral formulations are described in Akers, et al. J. Parenteral Sci. Tech. 1987 41:88-96; Nash, R A “Suspensions” in Encyclopedia of Pharmaceutical Technology 2^{nd} ed. Taylor and Francis, 2006, pp 3597-3610 (hereby specifically incorporated by reference in the present application).

A controlled “flocculated suspension” as used here is a physically stable suspension of loosely aggregated particles of an active pharmaceutical ingredient such as a F1C that are microscopically separated. Such a suspension will usually settle in a loosely packed scaffold-like structure that is easily redispersed to reform the original suspension. This is in comparison to a non-flocculated suspension, which typically forms a hard cake that is more difficult to redisperse. The particles of a flocculated suspension are separated, with a surfactant as an intermediary, at a distance that is reflective of a potential energy minimum interaction between the particles.

A “surface-active agent” (surfactant) is a substance, which, at low concentrations, interacts between the surfaces of immiscible liquids of an emulsion to alter the interfacial tension and thus will stabilize the emulsion or interacts between the surface of a particle and the surrounding liquid to improve suspendability. Surface-active agents are amphipathic in structure having both polar (hydrophilic) and non-polar (hydrophobic) regions in the same molecule. Examples of surface active agents used in the formulation arts are given in Corrigan, O. I.; Healy, A. M. “Surfactants in Pharmaceutical Products and Systems” in Encyclopedia of Pharmaceutical Technology 2^{nd} ed. Taylor and Francis, 2006, pp 3583-3596.

A “suspending agent” as used here is a substance that facilitates and maintains the physical stability of a suspension by adjusting the viscosity of the liquid component and to more closely match the density of this component with the density of the particles in the suspension such that sedimentation or separation is retarded. Non-limiting examples of suspending agents suitable for parenteral administration include cellulose and derivatives thereof, such as sodium carboxymethylcellulose (CMC), methylcellulose microcrystalline cellulose, and dextran and derivatives thereof, gums, clays and gelatin. For injection dependent routes of administration of suspensions, CMC or gelatin are typically used. Considerations for choice of a suitable suspending agent include resuspendability of the drug in the diluent or vehicle to permit homogeneous dosing when withdrawing the suspension from its container or packaging system, avoidance of a physically instability (e.g. hard caking), syringeability, which is the ability to withdraw a homogeneous dose of the composition or formulation from its container or packaging system and injectability, which is the ability to eject the composition or formulation through the needle used to administer the composition or formulation to a subject.

“Flocculating agent” as used here is a substance that links particles of an active pharmaceutical ingredient such as a F1C into loose aggregates to form a flocculated suspension and includes ionic and amphoteric surfactants, hydrophilic polymers, clays and electrolytes. Considerations for the choice of a suitable flocculating agent include those given for a suspending agent. Additionally, an ionic or amphoteric flocculating agent modifies the charge on the surface of a particle in order to provide a zeta potential in the liquid media that allows the particles of the suspension to loosely aggregate.

A “wetting agent” as used herein is a surfactant and permits interaction between a particle of an active pharmaceutical ingredient such as a F1C that has a hydrophobic surface and an aqueous-based solution. Typically, in a suspension the hydrophobic surface is due to a F1C that is insoluble in the aqueous-based diluent or vehicle used to form or reconstitute the suspension.

An “emulsion” as used here is a mixture comprising an active pharmaceutical ingredient such as a F1C and an oil- and water-based diluent or vehicle and one or more surface-active agents that facilitate and maintain the oil-in water phase. Emulsions typically contain a surfactant (emulsifier) and a co-surfactant (co-emulsifier). The co-surfactant (or “co-emulsifier”) is typically a polyglycerol derivative, a glycerol derivative or a fatty alcohol. Typical emulsifier/co-emulsifier combinations by way of example and not limitation are glyceryl monostearate and polyoxyethylene stearate; polyethylene glycol and ethylene glycol palmitostearate; and caprilic and capric triglycerides and oleoyl macrogolglycerides. The aqueous phase includes water, buffers, glucose, propylene glycol, polyethylene glycols (PEGs), typically of lower molecular weight (e.g. PEG 300 or PEG400), and glycerol. The oil phase includes fatty acid esters, modified vegetable oils, mixtures of mono- di- and triglycerides and mono- or di-esters of PEG. Examples of emulsions and their preparation are provided by Eccleston, G. M. “Emulsions and Microemulsions” in Encyclopedia of Pharmaceutical Technology 2^{nd} ed. Taylor and Francis, 2006, pp 1548-1565 (hereby specifically incorporated by reference in the present application).

A “diluent”, as used here, typically includes a non-aqueous liquid, such as benzyl benzoate, cottonseed oil, N,N-dimethylacetamide, a C₂₋₁₂ alcohol (e.g., ethanol), glycerol, peanut oil, propylene glycol, a polyethylene glycol (“PEG”), vitamin E, poppy seed oil, propylene glycol, safflower oil, sesame oil, soybean oil and vegetable oil or an aqueous liquid, such as WFI (water for injection) or D5W (5% dextrose in water for injection) that may include one or more other excipients such as buffers, chelating agents and preservatives. A diluent may also comprise a mixture of aqueous and water-miscible liquids.

A “vehicle” as used here is a diluent(s) that comprises the majority of the total volume or mass of an invention composition or formulation to be administered parenterally.

“Aqueous-based” as used here means a diluent, vehicle, or a solution wherein the major component by volume is water.

An “air oxidizable excipient” as used herein is an excipient that may form one or more degradants attributable to exposure of the excipient to oxygen or air, either alone or when blended into an invention composition or formulation, at elevated, ambient or storage temperatures or contains a contaminate or a degradant from synthesis or in a commercial preparation that is subject to degradation on exposure to oxygen or air.

An “excipient degradant” is a substance derived from a chemical breakdown of an excipient (i.e., an air oxidizable excipient) resulting from its exposure to oxygen or air. The excipient degradant may be a degradant that is a direct consequence of the breakdown of the excipient or may be produced from subsequent interaction(s) of the initially formed degradant with another excipient, the F1C, or with water or oxygen that is dissolved in the invention composition or formulation or is present in air to which the invention composition of formulation is exposed. An excipient degradant resulting from oxidation, at elevated, ambient or storage temperatures, of an air oxidizable excipient, either alone or when it is blended into an invention composition or formulation, will typically have one or more oxygen atoms derived from dissolved oxygen in a suspension formulation and one or more carbon atoms derived from the air oxidizable excipient. An excipient degradant may also be initially present as an impurity in a commercially available excipient used in preparation of the suspension formulation.

A “destabilizing excipient degradant” is an excipient degradant or an impurity that adversely changes the efficacy of an invention composition or formulation or occurs concomitantly with a loss of efficacy. An adverse change in efficacy is a reduction of 5 to about 100% in a desired pharmacological response in a subject as compared to an otherwise substantially identical composition or formulation that is absent the destabilizing excipient degradant. Typically the reduction is 50 to about 100% or results in a transformation of a pharmaceutically acceptable formulation or composition to one that is no longer efficacious for treating the intended condition. In some embodiments the destabilizing excipient degradant results from air oxidation of the excipient. In other embodiments the destabilizing excipient degradant is an impurity in a commercial product.

“Heavy metal” includes one, two or more metals selected from the group consisting of as iron, selenium, manganese, copper, zinc, cobalt, lead, arsenic, aluminum, nickel, tin, niobium, molybdenum, titanium, vanadium and chromium in zero or positive oxidation states, typically from +1 to +4 with simultaneous presence of one or more, typically one or two, being contemplated. For example, iron may be present in zero, +2 or +3 oxidations states or in a combination of such states and copper may be present in zero, +1 or +2 oxidation states or a combination thereof. A particular subset of heavy metals includes one, two or more metals that are capable of supporting Fenton chemistry (i.e., generates reactive oxygen species upon interaction with molecular oxygen or peroxide) and include metals ions such as Fe²⁺, Cu¹⁺, Co²⁺, Ti³⁺, V²⁺, and Cr³⁺ ions. These and other heavy metals capable of supporting Fenton-type chemistry are discussed in Goldstein S., et al. “The Fenton reagents” Free Radical Biol. Med. 15: 435-445, 1993, which is incorporated by reference with Table 2 of page 440 particularly incorporated by reference. Sources of heavy metal contamination include interaction of an excipient or F1C with a stainless steel container(s) used in preparation of an invention composition or formulation described herein. Another source of contamination is heavy metal already present in an F1C or an excipient prior to preparing an invention composition or formulation described herein. For example, a therapeutically acceptable excipient may have heavy metal content as measured by sulfide precipitation that is equivalent to the presence of ppm of lead (expressed as ppm lead equivalent) of up to 20 ppm, which is the limit acceptable for some NF grade excipients. For example, lots of commercially available sodium phosphate monobasic anhydrous and sodium phosphate dibasic anhydrous meeting NF specifications used in preparing a buffered diluent may have 10 and 20 ppm lead equivalents, respectively. Yet another source of heavy metal contamination may come from a container system containing a dosage form of a invention composition or formulation due to leaching for example from the glass, septum or other parts of the system used to isolate the invention composition or formulation from its surroundings.

“Essentially free” as used here means a component of an invention composition or formulation or the composition or formulation itself, so defined, that does not contain an impurity or degradant derived from or is due to an air oxidizable excipient in an amount that measurably reduces the efficacy of the composition or formulation for its intended purpose(s) or is associated with measurably reduced efficacy.

“Effective amount” as used herein in a content of describing an amount of an excipient means an amount of an excipient that will provide the desired property or properties of the excipient without interfering to a measurable extent the desired pharmacological properties of the active pharmaceutical ingredient or other excipients in a composition or formulation.

“Therapeutically effective amount” as used here is an amount of an invention composition or formulation that contains sufficient pharmaceutically active ingredient and has acceptable toxicity in relation to the condition being treated but has sufficient efficacy as contained within the composition or formulation to elicit the desired therapeutic effect after administration of the composition or formulation to a subject through an intended route of administration. In the context of treating an immune suppressive condition or an unwanted immune condition it is the amount sufficient to restore normal or improve immune responsiveness in an immunodeficient subject to which it is administered or to detectably modulate or improve an immune or cellular parameter or symptom. Such modulation or improvement is consistent with either restoring or enhancing a desired immune response, with inhibiting the progression of the disorder or with inhibiting the replication of a pathogen. Immune and cellular parameters that may be detectably improved include, e.g., (1) increased expression or biological activity of one or more Th1 associated cytokine, interleukin, growth factor, enzyme or transcription factor, (2) decreased expression or biological activity of one or more Th2 associated cytokine, interleukin, growth factor, enzyme or transcription factor, (3) decreased expression or biological activity of one or more inflammation associated cytokine, interleukin, growth factor, enzyme or transcription factor and (4) inhibition of the replication of a pathogen such as a virus or bacterium or pathological cell or cell type such as an infected cell, a malignant cell or cancer cell. The immune and cellular parameters that are detectably improved may be improved due to direct or indirect effects of an active pharmaceutical ingredient such as a formula 1 compound.

Thus, a therapeutically effective amount is an amount of an active pharmaceutical ingredient in an efficacy stabilized composition or formulation that is sufficient for treatment, prevention or amelioration of the infection, immune suppression, unwanted immune response, blood deficiency disorder or radiation exposure or other condition or symptom being treated. Amelioration of a disease or a symptom may be determined subjectively or objectively, e.g., by the subject or by conducting an appropriate assay or measurement such as one described herein. A dosage of a composition or formulation given herein, therefore, refers to the equivalent weight of the active pharmaceutical ingredient in its unionized form that is present in the composition or formulation, as is common practice in the formulation arts. The volume of a solution or suspension composition or formulation to be parenterally administered therefore is dependent on the concentration of the active pharmaceutical ingredient in the composition or formulation just prior to its administration (i.e., after addition of any required diluent or vehicle).

“Preventing” or “prevention” as used herein has the meaning commonly apply to the medical arts and thus means taking advance measures against a condition or disease state that is possible or probable or defending against a condition. Therefore preventing or prevention does not mean only or is not restricted to stopping each and every conceivable occurrence of a condition so referenced with certainty.

“Prophylactic” as used herein means defending against a disease and does not mean stopping the occurrence of a condition so referenced under every conceivable circumstance with certainty.

“Subject to developing” as used herein means prone to, at risk of, or tending towards developing a condition so referenced.

“Condition”, “disease” or “disease state” as used herein are interchangeable terms and refers to a physiological state in a subject that is not normal or is abnormal in intensity or duration and can be treated or prevented by administration of an invention composition or formulation.

“Peroxide value” as used herein means an amount of peroxide in a peroxide-containing compound that is equivalent to that same amount of hydrogen peroxide or oxygen in its ability to oxidize a substrate. Peroxide values (PV) are given in mequiv or μequiv per unit weight or per unit volume of a test article (excipient, suspension formulation, solution formulation, etc.).

An “oxygen-depleted atmosphere” as used herein means an atmosphere that contains a partial pressure of oxygen in about 0.1 to about 0.03 bar or less, typically in about 0.1 to about 0.3 bar, more typically in about 0.3 bar. Alternatively, the internal atmosphere contains less than 10% oxygen, less than 5% oxygen, less than 2.5% oxygen or consists essentially of an inert gas such as nitrogen.

“Insoluble” as used here means a property of an active pharmaceutical ingredient such as a F1C so defined wherein the compound so referenced is poorly soluble in a specified liquid, typically a pharmaceutically acceptable diluent used as a component in a solution composition or formulation for parenteral administration. A substance is typically considered insoluble in a solvent when the concentration dissolvable in a defined solvent at ambient temperature is about 100 μg/mL or less.

A “reactive oxygen species” as used here is a reactive species that includes singlet oxygen, hydrogen peroxide, hydroxyl radical, peroxides, hydroperoxides, acylperoxyacids, peroxyl radicals, acylperoxyl radicals, alkoxyradicals, and any other reactive species having a —O—O— functional group or an oxygen-based unpaired electron.

A “metal chelator agent” or “heavy metal chelator agent” as used here is a substance that sequesters heavy metals by binding to the metal through two or more complexing groups from the same molecule (i.e. chelator agent). Examples of pharmaceutically acceptable metal chelators by way of illustration and not limitation are ethylenediaminetetraacetic acid (EDTA), ethyleneglycoltetraacetic acid (EGTA), diethylene-triaminepentaacetate (DTPA), hydroxyethylethylene-diaminetriacetic acid (HEEDTA), diaminocyclohexane-tetraacetic acid (CDTA) or 1,2-bis(2-aminophenoxy)ethane-N,N,N′,N′-tetraacetic acid (BAPTA). In the pharmaceutical arts, EDTA or a derivative thereof is referred to as “edetate” while a DTPA or a derivative thereof is referred to as “pentetate”. EDTA derivatives typically employed include its pharmaceutically acceptable salts such as trisodium edetate, tetrasodium edetate and disodium calcium edetate. Suitable DTPA salts are similarly named.

A “free radical inhibitor” as used here is a substance that prevents or retards formation, propagation or reactions of a free radical or otherwise suppresses the auto-oxidation of a substance to be protected. Free radical inhibitors include antioxidants and metal chelator agents, such as an edetate, a pentetate and the like. Metal chelator agents retard the formation of radicals through binding of heavy metal that may serve as a catalyst for the formation of the radical, and antioxidants terminate the propagation of radicals, and include by way of example and not limitation, ascorbic acid, tartaric acid, malic acid, fumaric acid, glutamic acid, propyl gallate, sodium metabisulfite, dithiothreitol, carotenes, tocopherols, plant phenols and other phenols such as butylated hydroxytoluene and butylated hydroxyanisole. These and other antioxidants are described in Waterman, K. C., et al. “Stabilization of pharmaceuticals to oxidative degradation” Pharm. Dev. Tech. 7(1): 1-32, 2002, which is incorporated by reference herein with Table 12 of page 26 particularly incorporated by reference.

The term “radiation therapy” or “radiotherapy” as used here refers to use of high-energy radiation to treat cancer. Radiation therapy includes externally administered radiation, e.g., external beam radiation therapy from a linear accelerator, and brachytherapy, in which the source of irradiation is placed close to the surface of the body or within a body cavity. Common radioisotopes used include but are not limited to cesium (Cs¹³⁷), cobalt (Co⁶⁰), iodine (I¹³¹), phosphorus-32 (P³²), gold-198 (Au¹⁹⁸), iridium-192 (Ir¹⁹²), yttrium-90 (Y⁹⁰), and palladium-109 (Pd¹⁰⁹). Radiation is generally measured in Gray units (Gy), where 1 Gy=100 rads.

The term “radiation exposure” as used here refers to a subject experiencing ionizing radiation due to radiation therapy or from dispersion of nuclear material.

“Immune suppressive condition” as used herein is a condition characterized by an absence of and/or an inadequate degree a desired Th1 immune response that is normally provided to a disease challenge. Deficient Th1 immune responses and their treatments are given in Ahlem, et al. U.S. Pat No. 6,667,299 (hereby specifically incorporated by reference into the present application).

“Blood cell deficiency” as used herein is a condition or symptom due to one or more hematopoietic cell types being present in abnormal amount(s) such as in thrombocytopenia and neutropenia. Thrombocytopenia (“TP”), abnormally low platelet counts, can arise from impaired platelet production, sequestration of platelets in the spleen or abnormal loss of circulating platelets. Impaired production can result from causes such as chemotherapies or radiation therapies. Abnormal loss of circulating platelets is often associated with autoreactive antibodies that bind to platelets and reduce their life span. These underlying causes give rise to the various clinical forms of TP, such as autoimmune neonatal TP, immune thrombocytopenic purpra, radiation induced TP, chemotherapy induced TP and amegakaryocitic TP. Neutropenia (“NP”), is considered to exist clinically when neutrophils drop to below a level considered normal. NP can arise from impaired production of neutrophil precursors or mature neutrophils, movement of neutrophils from the circulation to tissue, abnormal circulating neutrophil loss or a combination of these causes. Impaired neutrophil production can be acquired from, e.g., treatment with a cytotoxic or cytostatic drug, chemotherapy, radiation therapy or an autoimmune response. The abnormal loss of circulating neutrophils in autoimmunity is associated with autoreactive antibodies that bind to the cells and reduce their life span. These underlying causes give rise to the various clinical forms of NP, such as postinfectious NP, drug-induced NP, autoimmune NP, or chronic idiopathic NP.

### INVENTION EMBODIMENTS

In one embodiment the invention compositions and formulations are aqueous-based suspensions that can be used for parenteral administration of an active pharmaceutical ingredient, such as a Formula 1 Compound (F1C), that is insoluble in water to a subject in need thereof. F1C compounds include androst-5-ene-3β,17β-diol, androst-5-ene-3β,7β,17β-triol and their ester and ethers derivatives such as 3β-acetoxy-androst-5-ene-17β-diol, 3β-acetoxy-androst-5-ene-7β,17β-diol, 3β-methoxy-androst-5-ene-17β-diol, 3β-methoxy-androst-5-ene-7β,17β-diol and their corresponding propionate esters or ethoxy ethers.

It has been unexpectedly found that reduction in efficacy of a composition or formulation comprising an F1C and an air oxidizable excipient is uncoupled from transformation of a pharmaceutically acceptable to a pharmaceutically unacceptable composition or formulation due to a decrease in F1C strength (i.e. decrease in the mass amount of F1C in the composition or formulation), pH, color change, suspendability, syringeability, particulate content, or any other physiochemical characteristics normally associated with pharmaceutical acceptability although such changes may occur concomitantly. Thus, a loss of efficacy results even though the F1C strength may remain within label (i.e., 95-105% of nominal) or is decreased to an amount insufficient to account for the observed loss of efficacy. Furthermore, a non-efficacy composition or formulation may be stabilized with respect to a physiochemical parameter, such as pH or suspendability through for example the inclusion of a pH stabilizer, yet may still provide a unsuitable composition or formulation due to loss of efficacy. Thus, invention embodiments provide efficacy stabilized compositions and formulations that are stabilized with respect to pH, but would have otherwise remained subject to loss of efficacy due to the presence of a destabilizing excipient degradant or formation of too much of a degradant from interaction of an air oxidizable excipient with molecular oxygen, either prior to or after blending of the air oxidizable excipient into the invention composition or formulation.

Compositions and formulations for parenteral administration will usually employ a vehicle as a liquid diluent that provides, by way of example and not limitation, a liquid solution for intravenous injection (i.v.) or a suspension for introduction of an active pharmaceutical ingredient such as a F1C for intramuscular (i.m.) or subcutaneous (s.c.) injection for introduction to a subject of a steroid drug, hydroxy steroid, glucocorticoid or a F1C. Alternatively, the vehicle may be an oil which forms a solution, suspension or emulsion, that is suitable for non-intravenous routes of parenteral administration, or which form a solution, suspension, emulsion, gel or cream that is suitable for non-injection dependent routes of parenteral administration.

Invention compositions or formulation as dry powders or lyophilized solids are also contemplated with parenteral administration occurring after introduction of a vehicle to the dry powder or lyophilized solid (for reconstitution to a solution or suspension formulation). Dry powder formulations and devices for pulmonary delivery are given in Donnelly, U.S. Pat. No. 6,878,751 (hereby specifically incorporated by reference into the present application). Lyophilized formulations used in parenteral delivery of active pharmaceutical ingredient as a suspension are given in Geller, et al. U.S. Pat. No. 5,002,940 (hereby specifically incorporated by reference into the present application). The principal advantage of a dry powder or lyophilized composition or formulation is the stability of an oxidizable excipient that is employed is usually improved as compared to a solution or suspension dosage form due to the absence of prolonged contact with a vehicle or diluent on storage, which would otherwise promote degradation of the air oxidizable excipient and subsequent reaction of the degradant(s) so derived with the active pharmaceutical ingredient or with another component of the composition or formulation. Although stability of invention compositions or formulations may be improved with respect to some characteristics, such as pH stability, using a solid dosage form, efficacy stability of the composition or formulation will nonetheless require practice of the invention(s) disclosed herein.

Appropriateness of a particular dosage form for parenteral of an invention composition or formation or will be dependent, among other considerations, on the intended route of administration or the desirability or undesirability of sustained release of a F1C contained within the composition or formulation from the site of administration. For example, sustained release from intramuscular, intradermal or subcutaneous injection of a suspension or emulsion would be appropriate if prolonged response to a F1C is desired. Examples of parental dosage forms and delivery systems are found in The Merck Veterinary Manual 50^{th} ed. Merck and Co., Inc. Whitehouse Station, N.J., 2006 (hereby specifically incorporated by reference into the present application).

An exemplary solution for injection is a mixture of 2 or more components (ingredients) that form a single phase that is substantially homogeneous down to the molecular level with the exception the solution may contain a pharmaceutically acceptable level of foreign particulates. “Water for injection” is the most widely used diluent or vehicle for parenteral formulations. However, a nonaqueous solvent or a mixed aqueous/nonaqueous solvent system may be necessary to stabilize drugs that are readily hydrolyzed by water or to improve solubility. A range of excipients may be included in parenteral solutions, including antioxidants, antimicrobial agents (preservatives), buffers, chelating agents, inert gases, and substances for adjusting tonicity, one or more of which may be dissolved within a vehicle or diluent or may be present in a invention composition or formulation to which the diluent or vehicle is added. Antioxidants maintain product stability by being preferentially oxidized over the shelf life of the product. Antioxidants that are free radical inhibitors slow the rate of an auto oxidation process by obviating the reactivity of a free radical that initiates or propagates the auto oxidation process. Antimicrobial preservatives inhibit the growth of any microbes that are accidentally introduced while doses are being withdrawn from multiple-dose bottles and act as adjuncts in aseptic processing of products. Buffers are used to maintain solubility of the active ingredient or stability of the composition or formulation. Metal chelating agents are added to complex and thereby inactivate metals, including copper, iron, and zinc, and various ions thereof, that can catalyze oxidative degradation of an oxidizable excipient. Inert gases are used to displace air dissolved in solutions or suspensions or which is in the headspace of containers or dosage forms that contain the composition or formulation in order enhance product integrity of oxygen-sensitive excipients (i.e. air oxidizable excipients). Isotonicity of the formulation is achieved by including a tonicity-adjusting agent. Addition of a tonicity agent provides an injectable composition or formulation that has substantially the same osmotic pressure as blood. Failing to adjust the tonicity of the solution can result in the hemolysis or crenation of erythrocytes when hypotonic or hypertonic solutions, respectively, are given IV in quantities >100 mL. Injectable compositions or formulations must be sterile and free of pyrogens. Pyrogenic substances are primarily lipid polysaccharides derived from microorganisms, with those produced by gram-negative bacilli generally being most potent. Injectable solutions are commonly used in parenteral administration and such solutions given IM or subcutaneously result in rapid drug absorption, provided precipitation at the injection site does not occur.

A suspension for injection consists of insoluble solid particles dispersed in a liquid medium, with the solid particles accounting for about 0.1 to 50% w/v, typically 0.5-30% w/v of the suspension. The vehicle may be aqueous based, oil based, or both. Caking of injectable suspensions is minimized through the production of flocculated systems, which are comprised of clusters of particles (flocs) held together in a loose open structure. Excipients, other than a diluent, that are commonly used in invention compositions and formulations for blending into a suspension dosage form include suspension agents, wetting agents, flocculating agents, surface-active agents, buffering agents, heavy metal chelator agents, antioxidants and preservatives. In one embodiment, a composition or formulation suspension will contain at least one surface-active agent (surfactant), typically one or two, and one or more other excipients listed immediately above. Oftentimes an excipient will serve more than one purpose. By way of example and not limitation, a surface-active agent may act as a suspending/flocculating agent, a wetting agent or may serve both purposes in an invention composition or formulation. Additionally, a metal chelator agent may be used in an invention composition or formulation as a pH stabilizer that will also have anti-microbial properties and thus will serve as a preservative in whole or in part.

Compared with that of injectable solutions, the rate of drug absorption from injectable suspensions can be prolonged because additional time is required for disintegration and dissolution of the drug particles. The slower release of drug from an oily suspension compared with that of an aqueous suspension is attributed to the additional time taken by drug particles suspended in an oil depot to reach the oil/water boundary and become wetted before dissolving in tissue fluids. This phenomenon of delayed release from aqueous or non-aqueous formulation is often referred to as the “depot” effect. The ease of injection and the availability of the drug in depot therapy, which exploits the depot effect, are affected by the viscosity of the suspension and the particle size of the suspended drug. These systems can afford enhanced stability to active ingredients that are prone for example to hydrolysis in aqueous solutions.

Suspensions will typically employ a surface-active agent (surfactant). One function of a surfactant may be to wet the suspended powders (i.e., surfactant used as a wetting agent) and provide acceptable syringeability. Another role of the surfactant may be to aggregate the particles in a floc, which aids in resuspendability. In some embodiments, both a wetting agent and a flocculating agent are used in a suspension dosage from. In other embodiment, a single surfactant will play both roles. A suspending agent may also be used in a suspension composition or formulation in order to modify the viscosity of the formulation, so as to retard settling of the particles into a hard cake. Suitable surfactants include anionic, nonionic (amphoteric), cationic and zwitterionic surfactants. In one embodiment the suspension is an aqueous-based flocculated suspension. Guidance for the preparation of flocculent suspensions is given in Nash, et al. U.S. Pat No. 3,457,348 (hereby specifically incorporated by reference into the present application).

Examples of nonionic surfactants include, but are not limited to, the following: 1) Reaction products of a natural or hydrogenated castor oil and ethylene oxide. The natural or hydrogenated castor oil may be reacted with ethylene oxide in a molar ratio of from about 1:35 to about 1:60, with optional removal of the PEG component from the products. The PEG-hydrogenated castor oils, available under the trademark CREMOPHOR, are examples; 2) Polyoxyethylene-sorbitan-fatty acid esters, also called polysorbates, e.g., mono- and tri-lauryl, palmityl, stearyl and oleyl esters of the type known and commercially-available under the trademark TWEEN, including Tween 20 [polyoxyethylene(20)sorbitan monolaurate], Tween 40 [polyoxyethylene(20)sorbitan monopalmitate], Tween 60 [polyoxyethylene(20)sorbitan monostearate], Tween 65 [polyoxyethylene(20)sorbitan tristearate], Tween 80 [polyoxyethylene(20)sorbitan monooleate], Tween 81 [polyoxyethylene(5)sorbitan monooleate] and Tween 85 [polyoxyethylene(20)sorbitan trioleate]. In some embodiments the nonionic surfactant is Polysorbate 80 or Polyoxyethylene (20) sorbitan monooleate that has been selected from a commercial source or purified to minimize introduction of impurities into a composition or formulation that would result in a non-efficacy stabilized composition or formulation if not subsequently removed after blending into the invention composition or formulation.

Other non-ionic surfactants include 1) Polyoxyethylene-polyoxypropylene block polymers (sometimes referred to as poloxamers) and are composed of a central hydrophobic chain of polyoxypropylene flanked by two hydrophilic chains of polyoxyethylene and have a molecular weight ranging from about 2000 to about 15,000 daltons and have the general formula: HO(CH₂O₄)ₐ—(CH₃.O₆)_{b}—(CH₂O₄)ₐ—H wherein a is about 10 to about 150, representing blocks of repeat units of polyethylene oxide or polyoxyethylene and b is about 20 to about 60, representing blocks of repeat units of polypropylene oxide or polyoxypropylene. Uses of poloxamers as surface-active agents are described in Smithy, DT US Pat Appl No. 2007/0141143 (hereby specifically incorporated by reference into the present application). Poloxamers are commercially available under the trademark PLURONIC, EMKALYX and POLOXAMER, 2) Dioctylsulfosuccinate or di-[2-ethylhexyl]-succinate, 2) PEG mono- and di-fatty acid esters, such as PEG dicaprylate, also known and commercially-available under the trademark MIGLYOL 840, PEG dilaurate, PEG hydroxystearate, PEG isostearate, PEG laurate, PEG ricinoleate, and PEG stearate, 3) Polyoxyethylene alkyl ethers, such as those commercially-available under the trademark BRIJ, e.g., Brij 92V and Brij 35, polyoxy 10 stearyl ether, poloxy 20 stearyl ether, 4) Fatty acid monoglycerides, e.g., glycerol monostearate and glycerol monolaurate, glycerol monopalmitate, glycerol monooleate, glycerol monocaprylate, 5) Tocopherol esters, e.g., tocopheryl acetate and tocopheryl acid succinate and 6) Succinate esters, e.g., dioctylsulfosuccinate or related compounds, such as di-[2-ethylhexyl]-succinate

Examples of anionic surfactants include, but are not limited to, sulfosuccinates, phosphates, sulfates and sulfonates. Specific examples of anionic surfactants are sodium lauryl sulfate, ammonium lauryl sulfate, ammonium stearate, alpha olefin sulfonate, ammonium laureth sulfate, ammonium laureth ether sulfate, ammonium stearate, sodium laureth sulfate, sodium octyl sulfate, sodium sulfonate, sodium sulfosuccinimate, sodium tridecyl ether sulfate and triethanolamine lauryl sulfate.

Examples of cationic surfactants include but are not limited to palmitoyl DL camitine chloride, cetylpyridinium chloride, dimethylammonium and trimethylammonium surfactants of chain length from 8 to 20 and with chloride, bromide or sulfate counterion, myristyl-gammapicolinium chloride and relatives with alkyl chain lengths from 8 to 18, benzalkonium benzoate, double-tailed quaternary ammonium surfactants with chain lengths between 8 and 18 carbons and bromide, chloride or sulfate counterions. Other pharmaceutically acceptable surfactants are given in Anderson, A U.S. Pat. No. 6,991,809 (hereby specifically incorporated by reference into the present application).

Typically used surfactants for injectables include benzalkonium chloride, sodium deoxycholate, myristyl-γ-picolinium chloride, Poloxamer 188 (a Polyoxyethylene-polyoxypropylene block polymer with formula HO(C₂H₄O)ₐ(C₃H₆O)b(C₂H₄O)ₐH and average M.W. 8400, polyoxyl castor oil and related PEGylated castor oil derivatives such as Cremophor EL, Arlatone G (polyoxyethylene (25) hydrogenated castor oil), sorbitan monopalmitate, Pluronic 123 (block polymer of the formula [(EO)₂₀(PO)₇₀(EO)₂₀], wherein EO is an ethyeneoxy subunit and PO is propyleneoxy subunit), sodium 2-ethylhexanoic acid and polyoxyethylene-sorbitan-fatty acid esters (polysorbates).

A suspension of an active pharmaceutical ingredient such as a F1C is typically used when the API insoluble or has insufficient solubility in a pharmaceutically acceptable diluent or vehicle. To have more consistent systemic exposure of an active pharmaceutical ingredient administered as a suspension dosage form, the F1C is usually “micronized”. Micronization may be accomplished by mechanical milling, ultrasonic disintegration, microfluidization, melt extrusion, spray drying, spray freeze-drying or precipitation. Micronization techniques are described in Drug Delivery Technology 2006, 6:54-60; Serajuddin, A T M J. Pharm. Sci. 1999, 88:1058-1066 (hereby specifically incorporated by reference into the present application). The active pharmaceutical ingredient may be micronized separately or co-micronized with a surface-active agent, wetting agent or other carrier. Typically, the active pharmaceutical ingredient in micronized form is present in an active ingredient range between about 0.1-50% w/v of a suspension, typically 0.5 to 30% more typically about 10% by weight or 90.0 to 110.0 mg/mL.

Particle size, unless otherwise specified, refers to a number or volume weighted mean diameter. Oftentimes the particle size will be associated with a volume-weighted distribution known as a mean volume diameter and thus the particle size will be the diameter of particles, within a stated fraction (Dv) in a volume-weighted distribution of particles that will have the stated diameter. For example, a particle diameter represented by 35 μm (Dv, 0.90) means that 90% or more of the mass of particles will have a diameter of 35 μm or less. Particle size distribution is typically determined from laser beam scattering diffraction. Methods for determining particle size distribution and other techniques for describing liquid dispersions are given in Tinke, A P in “Particle Size and Shape Characterization of Nano- and Submicron Liquid Dispersions” Amer. Pharm. Rev. September/October 2006 (hereby specifically incorporated by reference into the present application). Typically, particles in a suspension dosage will be in a mean diameter range between about 1 μm-60 μm, more typically about between about 10 μm-60 μm or about 35 μm. Typically, in an active pharmaceutical ingredient to be used in preparation of a suspension dosage, no more than 20% of the distribution is above the mean volume diameter (Dv, 0.80), more typically less than or equal to 10% of distribution is above the mean volume diameter (Dv, 0.90).

In one embodiment the active pharmaceutical ingredient to be blended in a suspension dosage form is about 10% weight by volume of the suspension wherein the active pharmaceutical ingredient has been micronized to a provide a distribution of particles at a stated mean volume diameter with Dv, 0.90. Oftentimes the mean volume diameter of the active pharmaceutical to be used in blending of a suspension dosage form will be less than a mean volume diameter of particles desired in the final suspension dosage form in order to account for aggregation. An active pharmaceutical ingredient, such as a F1C, to be used in blending of a suspension formulation will have for example a F1C mean diameter between about 0.1 μm to 60 μm, typically about 5 μm to 60 μm, more typically between about 5 μm to 20 μm. In one embodiment the F1C is 3β,17β-di-hydroxy-androst-5-ene with a particle size of about 10 μm (Dv, 0.90) obtained by jet milling. In another embodiment androst-5-ene-3β,17β-diol has a particle size between about 3-5 μm of Dv, 0.90, which is obtained by microfluidization. Microfluidization uses high pressure to force carrier fluid containing a hydrophobic active pharmaceutical ingredient that is insoluble in the carrier fluid into microchannels. Methods for microfluidization are described in Sharma U.S. Pat. No. 6,555,139 (hereby specifically incorporated by reference into the present application).

In one embodiment the invention composition or formulation is protected from loss of efficacy on storage due to air oxidation of the air oxidizable excipient or subsequent generation of destabilizing excipient degradants by limiting the exposure to oxygen by providing for an oxygen-depleted atmosphere that is in contact with the invention composition or formulation. This is done by packaging the composition or formulation so as to provide for a sealed vessel or packaging system with an internal atmosphere (i.e. headspace) substantially free of oxygen or has significantly less oxygen than external ambient air. The internal atmosphere depleted in oxygen content typically contains 10% less oxygen, 5% less oxygen or preferably 2.5% less oxygen or lower compared to ambient air. More typically, the internal atmosphere established within the sealed vessel will consist essentially of an inert gas such as Nitrogen, Argon or Helium or a combination thereof or about 99% of the internal atmosphere is Nitrogen. Typically, Nitrogen is used in purging of the headspace of a sealable vessel due to cost considerations although argon is sometimes preferred due to its higher density relative to ambient air. Typically the inert gas used to establish the oxygen-depleted internal atmosphere will have an oxygen content of less than 5 ppm. In one embodiment, ultra-high purity inert gas (less than 0.5 ppm O₂) is used. Sometimes the inert gas is introduced and maintained over the invention composition or formulation during filling and sealing of the sealable vessel or packaging system, is introduced to or maintained over the sealable vessel or packaging system immediately subsequent to sealing or is introduced by purging the atmosphere within the container or packaging system subsequent to filing and then maintained until immediately prior to sealing or until sealing is effected. In one embodiment the liquid solution or suspension used to fill the sealable vessel or packaging system has been purged as subsequently described for reduction of dissolved oxygen within a solution or suspension.

In another embodiment the composition or formulation is protected from loss of efficacy on storage due to air oxidation of the air oxidizable excipient and subsequent generation of destabilizing excipient degradants by limiting oxygen exposure by sparging, using an inert gas, of a liquid diluent prior to or after filling of a sealable vessel or packaging system or by sparging a suspension prepared by contacting the air oxidizable excipient with a liquid diluent, optionally in the presence of an active pharmaceutical ingredient, one or more other excipients or a combination thereof. Sparging or degassing is a technique whereby dissolved oxygen in a liquid solution or suspension is displaced by an inert gas. In one method, an inert gas, as described previously, is bubbled through a liquid solution or suspension, optionally with stirring, at a flow rate of, for example, 25 mL/sec for a time sufficient to provide for an oxygen depleted invention composition or formulation. If a surface active agent is present, a lower flow rate may be necessary to minimize foaming. Lower flow rates may also be necessary for sparging suspensions of API in diluent if the diluent has a vapor pressure low enough to cause sufficient loss with the higher flow rate sufficient to adversely affect the potency of the suspension (i.e., API content). Efficiency of this technique is highest for Helium; however the terminal dissolved oxygen content obtained by this method will be essentially the same using the less expensive Nitrogen gas. Alternatively, dissolved oxygen may be removed by one or more freeze thawing cycles under reduced pressure, typically at 1 mmHg or less. Other techniques for reducing he dissolved oxygen content include sonication under reduced pressure or boiling at ambient pressure or reduced pressure, typically at 10-20 mmHg; however, use of this technique after introduction of an API or excipient to a liquid diluent may suffer from loss of the API or excipient due thermal degradation or variability of potency (i.e., API content) due to loss of diluent. Methods for removing dissolved oxygen in aqueous-based solutions and dissolved oxygen content expected from practicing the aforementioned sparging or degassing techniques is given by Butler, I. B., et al. “Removal of dissolved oxygen from water: A comparison of four common techniques” Talanta 41(2): 211-215, 1994, which is incorporated by reference herein with Table 2 of page 212 particularly incorporated by reference.

In one embodiment nitrogen gas having sufficient purity with respect to O₂ content is passed through an invention composition or formulation at a rate sufficient to reach dissolved oxygen content of about 0.5-0.2 ppm for a period of about 5-60 min or about 0.3-0.2 ppm or about 30 min. In one embodiment dissolved oxygen content within an efficacy stabilized invention composition or formulation is between about 1.2-0.2 ppm. In another embodiment dissolved oxygen content within an efficacy stabilized invention composition is about 0.3 ppm. In another embodiment a method for obtaining a suspension formulation depleted in dissolved oxygen comprises the step of contacting freshly distilled water for injection with an F1C, optionally in the presence of one or more excipients. Methods for determining dissolved oxygen in aqueous-based solutions or suspensions include electrochemical methods that for example use an oxygen-sensitive electrode such as a polarographic Clark-type electrode or by titration methods such as the Winkler titration, which relies upon the measurement of iodine from oxidation of iodide (see for example Clesceri, L. S, et al. Ed. “Standard methods for the examination of water and waste water” 17^{th Ed.} 1989 and Hitchman, M. L. “Measurement of dissolved oxygen”, Wiley N.Y., 1978), but may be less suitable due to the alkaline conditions of this analysis.

For a solid dosage form of an invention composition for reconstitution into a solution or suspension, a lyophilized solid may be produced through a lyophilization cycle that uses a backflushing step with an inert gas. The packing system or container is then typically sealed under partial vacuum to give an internal atmosphere within the packaging system or container having a lower pressure relative to the external atmosphere to which the container system will be exposed during storage. The internal pressure over the composition or formulation using this method is lower than 0.1 bar, 0.05 bar, or about 0.03 bar, typically lower than about 0.03 bar.

Typical containers for storage of the invention compositions and formulations will limit the amount of water and air that reaches the materials contained therein. Typically, formulations are packaged in hermetically or induction sealed containers. The containers are usually induction sealed. Water permeation characteristics of containers have been described, e.g., Containers—Permeation, chapter, USP 23 <671>, United States Pharmacopeial Convention, Inc., 12601 Twinbrook Parkway, Rockville, Md. 20852, pp.: 1787 et seq. (1995) (hereby specifically incorporated by reference into the present application). Use of glass scored ampoules, sealed under inert atmosphere, provides a significant barrier to ambient air infiltration, but such a container system is limited to single use and requires glass that minimizes production of fragments that could contaminate the parenteral formulation contained therein. In one embodiment the headspace of the container or packaging system is oxygen-depleted by a method previously described or is under partial vacuum. The internal atmosphere typically is typically nitrogen or argon in a weight ratio to oxygen contaminate not less than about 10:1, 20:1 or 40:1, typically about 40:1. In one embodiment the internal atmosphere contains less than 10% oxygen, less than 5% oxygen, less than 2.5% oxygen or consists essentially of nitrogen.

Packaging systems that minimize the re-introduction or air, and particularly that of oxygen, are desirable, even more so when the dosage form is other than a dry powder. A container system that minimizes oxygen leakage into the container system will extend the shelf-life of the drug product by retaining efficacy of the invention composition or formulation for a longer period of time that otherwise would be lost sooner due to destabilizing excipient degradants derived from the oxidizable excipient. General considerations for container closure integrity of parenteral vials are given in Morton, D K J. Parenteral Sci. Technol. 1987, 41: 145-158 (hereby specifically incorporated by reference into the present application). Test suitable for evaluating the closure of dosage units containing the invention composition or formulations include the helium leak testing technique, the CO₂ tracer gas technique, the vacuum decay technique and the high voltage spark test. A helium leak rate greater than 10⁻⁶ cc/sec is considered a failure for closure integrity. Helium leak rates lower than 10⁻⁶ cc/sec are associated with acceptable microbial challenge results which sometimes correlates with infiltration rates of ambient air. Conventional seal integrity methods (i.e. dye leakage tests) are less desirable since they have been associated in the literature with leak rates of 10⁻³ cc/sec. Description of the helium leak test is given in Kirsch, et. al., PDA J. Pharm. Sci. & Tech., 1996, 51:187-194; Kirsch, et. al., Ibid. 1996, 51:195-202; Kirsch, et. al., Ibid. 1997, 51:203-207; Kirsch, et. al., Ibid. 1997, 51:195-207 (hereby specifically incorporated by reference into the present application).

Oxygen pressure in the headspace within a container or packaging system can be measured by any suitable method, for example using an electrochemical cell, (e.g., a Checkmate™ 9900 oxygen analyzer), by Raman spectroscopy, or using a photoelectric system for determining elemental composition of a medium. An illustrative method is described in more detail in International Patent Publication No. WO 96/02835 (hereby specifically incorporated by reference into the present application). Alternative methods are described by Bailey et al. (1980) Journal of the Parenteral Drug Association 34, 127-133, and by Powell et al. (1986), Analytical Chemistry 58, 2350-2352 (hereby specifically incorporated by reference into the present application).

Another method of limiting exposure of an invention formulation or composition to atmospheric oxygen is to select a container having a capacity such that percentage fill volume (i.e., the percentage of total capacity occupied by the formulation) is maximized, and headspace volume thereby minimized, to limit oxidative degradation of the air-oxidizable excipient. However, sufficient headspace must remain in the container after filling of a suspension composition or formulation to allow for effective agitation of the sealed container or packaging system to allow for resuspension before use. For a lyophilized solid dosage form, space must be allowed to accommodate introduction of the vehicle.

Accordingly, one embodiment of the invention is an article of manufacture comprising a sealed packaging system or container having substantially oxygen-impermeable walls and a substantially oxygen-impermeable seal, and having contained therewithin (a) an aqueous suspension suitable for parenteral administration for treatment of a condition in a subject, that comprises (i) a F1C in a effective therapeutic amount for treating a condition in a subject in a volume withdrawable from the sealed package or container (ii) one or more surface-active agents in an effective surface-active agent amount to provide controlled flocculation of the F1C and one or more other excipients, wherein at least one excipient is an air-oxidizable excipient, and (b) an oxygen-depleted atmosphere in the headspace overlying the composition. Another embodiment of the invention is an article of manufacture as described immediately above wherein the aqueous suspension additionally contains dissolved within the aqueous diluent an anti-oxidant or a metal chelator agent present within an effective anti-oxidant range or an effective metal chelator range.

Typically, the amount of a solution or suspension to be in a container or packaging system is calibrated to provide a single withdrawable dose. In this situation exposure of the composition or formulation to oxygen present in the external atmosphere is minimized after the first unsealing of the sealed container or packaging system. Suitable containers include single-dose vials and disposable pre-filled syringes. The container is usually a vial, typically a glass vial. Typically, the container will have a sufficient headspace volume to permit agitation by, for example, manual shaking or inversion for the purpose of re-suspending a flocculated suspension. More typically, the headspace occupies at least about 25%, or at least about 50% of the internal volume of the container. For androst-5-en-3β,17β-diol or androst-5-en-3β,7β-17β-triol suspension formulations will typically contain about 50 mg/mL to about 400 mg/mL of API in vehicle along with other excipients, e.g., a heavy metal chelating agent and a surface active agent. Administration intramuscularly (i.m.) of such formulations typically does not exceed a volume of 4 mL for a single injection. Typically, 1 mL volumes are employed for single i.m. injections. Typically, suspensions 50 mg/mL of API are in used for administration of androst-5-en-3β,17β-diol or androst-5-en-3β,7β-17β-triol.

Another method of limiting exposure of an invention formulation or composition to atmospheric oxygen, and thus improve shelf-life of the dosage form, is to decrease the concentration of dissolved oxygen in a liquid diluent or vehicle used to prepare a liquid composition or formulation. One method, as previously indicated for liquid and suspension compositions or formulations, is purging the diluent or vehicle with an inert gas by the methods of sparging or freeze-thawing. Another method is vacuum filtration of the diluent or vehicle, optionally followed by backflushing with an inert gas of the filtrate that is under partial vacuum. With aqueous-based diluents or vehicles, de-oxygenation may be accomplished by heating of the diluent or vehicle to its boiling point. Amounts of dissolved oxygen may be determined as previously described. Accordingly, in one embodiment for preparation of an efficacy stabilized suspension formulation, freshly prepared water for injection (example of diluent heated to its boiling point to effect de-oxygenation) is used to as the diluent.

In another embodiment the composition or formulation is protected from loss of efficacy on storage due to air oxidation of an air oxidizable excipient and subsequent generation of destabilizing excipient degradants by using one or more metal chelator agents, typically 1 or 2, preferably 1. Typically, the metal chelator agent is capable of sequestering heavy metals in media having a pH that is acceptable for a parenteral administration (i.e., not strongly acidic). Heavy metals to be sequestered include metal contaminates typically encountered in manufacturing processes that are capable of Fenton-type chemistry and include iron, copper or chromium.

An example of a suitable metal chelator agent is ethylenediamine-tetraacetic acid (EDTA), and pharmaceutically acceptable salts thereof, e.g. the pentasodium salt. Other suitable metal chelator agents by way of illustration and not limitation are ethyleneglycoltetraacetic acid (EGTA), diethylene-triaminepentaacetate (DTPA), hydroxyethylethylene-diaminetriacetic acid (HEEDTA), diaminocyclohexane-tetraacetic acid (CDTA), 1,2-bis(2-aminophenoxy)ethane-N,N,N′,N′-tetraacetic acid (BAPTA), nitrolotiacetic acid (NTA), ethylenediamine-bis-(o-hydroxyphenylacetic acid) (EDDA), and pharmaceutically acceptable salts thereof. Other classes of compounds that can be useful as chelating agents include polyfunctional acids such as citric acid and oxalic acid, amines such porphyrins, phenanthrolines, triethanolamine, and dimethylglyoxime and sulfur containing compounds such as 2,3-di-mercaptopropanol.

Metal chelator agent amounts that are used for efficacy stabilization include concentrations of about 0.002 to about 0.3% w/v, typically between about 0.01 to about 0.1%, more typically between about 0.01 to about 0.05% w/v. The heavy metal chelator agent may sequester heavy metals capable of Fenton-type chemistry that are initially present in the F1C or excipients, that are introduced during manufacturing of an invention composition or formulation or that leach out into the invention composition or formulation from a container system in which the invention composition or formulation is stored.

Without being bound by theory, binding of heavy metal contaminants by the heavy metal chelator agent may provide efficacy stabilization by reducing the availability of a heavy metal to generate reactive oxygen species (ROS) that are capable of oxidizing the air oxidizable excipient and thus producing one or more destabilizing excipient degradants. Production of ROS resulting from interaction of a heavy metal with molecular oxygen or peroxide is referred to as Fenton-type chemistry. Heavy metals capable of supporting Fenton-type chemistry in an aqueous based solution or suspension invention composition or formulation include iron. Fenton-type chemistry may be divided into two stages-initiation and propagation. In the initiation stage the heavy metal interacts with dissolved molecular oxygen or peroxide to form hydroxyl or peroxyl radical. The hydroxyl or peroxyl radical so formed then extracts a hydrogen atom from an air oxidizable excipient. The excipient radical then goes on to form excipient based peroxides that lead to formation of additional peroxyl radicals in the propagation stage, which accelerates destruction of the air oxidizable excipient.

Air oxidizable excipients particularly prone to destruction through Fenton-type chemistry contain a methylene or methine carbon directly adjacent to a heteroatom capable of stabilizing the radical resulting from hydrogen atom extraction from the methylene or methine carbon. Such excipients include those having subunits based on ethylene glycol such as a polyethelene glycol (PEG) or a polysorbate described herein. An end-stage result of Fenton-type chemistry on excipients having subunits based on ethylene glycol is production of acetaldehyde and formaldehyde than air oxidize further to the corresponding acid. Acid production from Fenton-type chemistry acting upon a susceptible excipient manifests itself as an increase in pH of a solution or suspension containing the excipient that eventually overwhelms the buffering capacity of the solution or suspension resulting in a decrease in pH to pharmaceutically unacceptable levels, which are typically below pH 4. However, it has been unexpectedly found that efficacy of formulations comprising an F1C and an air oxidizable excipient is lost prior to unacceptable pH excursions. Therefore, loss of efficacy of such formulations under these circumstances would be unexpected.

In consideration of the foregoing one embodiment to stabilize efficacy of an invention composition or formulation uses an effective amount of heavy metal chelator that may depend on heavy metal content in the invention composition or formulation and the strength of binding of the heavy metal chelator agent to heavy metals. When the heavy metal chelator agent is an edetate binding of the heavy metal is essentially irreversible. Thus, in one embodiment the amount of an edetate used in an efficacy stabilized invention composition or formulation is equal in molar amount to the iron content of the suspension formulation. This may be estimated by determining the lead equivalents, based upon sulfide precipitation, in a F1C and excipients used in preparing the invention composition or formulation. To specifically determine iron content, atomic absorption may be used. Typically, more than a minimum amount of metal chelator agent is used based upon initial heavy metal content to account for underestimations or heavy metal leaching from a container system in which the invention composition or formulation is stored. Typically, 5× the minimum amount is used.

In one embodiment, the metal chelator is an edetate present in an aqueous-based suspension of a F1C compound in a metal chelator agent amount of about 0.002 to about 0.3% w/v. In one embodiment the heavy metal chelator agent is one, two or more heavy metal chelator agents selected from the group consisting of an acid or pharmaceutically acceptable salt of ethylenediamine-tetraacetate (EDTA), ethyleneglycol-tetraacetate (EGTA), diethylenetriamine-pentaacetate (DTPA), hydroxyethylethylenediamine-triacetate (HEEDTA), diaminocyclohexane-tetraacetate (CDTA) or 1,2-bis(2-aminophenoxy)ethane-N,N,N′,N′-tetraacetate (BAPTA). In another embodiment the edetate is and acid or pharmaceutically acceptable salt of Ethylenediamine-tetraacetate of a combination thereof. In one embodiment an efficacy stabilized suspension formulation contains between about 0.01 to about 0.3% w/v EDTA, typically about 0.01 to about 0.05% w/v.

In another embodiment the invention composition or formulation is protected from loss of efficacy on storage by the presence of an excipient that is an antioxidant. Without being bound by theory, the role of an anti-oxidant is considered to be that of a free radical scavenger, which stabilizes efficacy of the composition or formulation. Non-limiting examples of suitable free radical scavenging antioxidants include butylated hydroxyanisole, butylated hydroxytoluene, propyl gallate, α-tocopherol (vitamin E), ascorbic acid (Vitamin C) and derivatives and salts thereof, including sodium ascorbate, ascorbic acid palmitate and erythorbic acid. Action of an antioxidant is sometimes sacrificial in that the antioxidant is destroyed upon scavenging the free radical. Because of its sacrificial nature, such free radical inhibitors will slow the propagation of a free-radical chain reaction (e.g., as in Fenton-type reactions) for a period of time, but are not typically used alone as the free radical inhibitor to limit degradation of an air-oxidizable excipient, but are typically used in conjunction with a non-sacrificial free radical inhibitor such a heavy metal chelator agent that inhibits initiation of radical chain reactions. Typical concentrations of antioxidants that may be used in invention compositions and formulations are 0.001-1.0 w/v %.

Other classes of compounds useful as anti-oxidants include thiols such as thioglycerol, cysteine, acetylcysteine, cystine, dithioerythreitol, dithiothreitol, gluthathione, sulfurous acid salts such as sodium sulfate, sodium bisulfite, acetone sodium bisulfite, sodium metabisulfite, sodium sulfite, sodium thiosulfate, sodium formaldehyde sulfoxylate and sodium thiosulfate.

Other compounds useful as anti-oxidants include butylated hydroxyanisole (BHA), butylated hydroxytoluene (BHT), fumaric acid and salts thereof, hypophosphorous acid, malic acid, alkyl gallates, for example, propyl gallate, octyl gallate and lauryl gallate and nordihydroguaiaretic acid.

Typically, an amount of an antioxidant optionally used in an invention composition or formulation is effective to substantially reduce formation of a degradant from auto-oxidation, typically in an antioxidant amount of about 0.001% to about 1% or about 0.01% to 0.1%.

A specific suitable metal chelator agent, optionally with a suitable antioxidant and their respective amounts to use with will a given active pharmaceutical ingredient will depend on such factor as the specific type of composition or formulation (solution, suspension, etc.) to be administered, the identity of other excipients to comprise the composition or formulation and the period of time over which efficacy is to be retained under specified storage conditions. The suspension formulations are thus stabilized for biological efficacy by reducing the rate of formation of unwanted degradants and-or limiting the potential for their spontaneous generation during storage.

One method to prepare an efficacy stabilized composition or formulation, which improves the shelf life of the composition or formulation, is to minimize the exposure of oxygen to the invention composition or formulation during preparation and filling. Another method slows the deleterious effect on efficacy due to action of residual oxygen remaining within an oxygen-depleted container or packaging system that holds the composition or formulation so effected by using a metal chelator agent or an antioxidant or both in combination. The deleterious effect of reactive oxygen species and destabilizing excipient degradants on efficacy is distinct from the effects these substances would have on the chemical stability of the active pharmaceutical ingredient or a physiochemical parameter traditionally employed to evaluate the pharmaceutical acceptability of a formulation such as pH, although beneficial effects on chemical stability of active pharmaceutical ingredient or on pH may occur concurrently through practice of the inventions disclosed herein.

One method for preparation of an efficacy stabilized composition or formulation, which improves the shelf life of the composition or formulation, is to minimize the initial burden that a composition or formulation contains of a reactive oxygen species or of a destabilizing excipient degradant. By way of example and not limitation, an air oxidizable excipient will contain a moiety derived from an unsaturated fatty acid or will contain a methine (—CH—) or methylene (—CH₂—) carbon directly attached to a heteroatom as found in an ethyleneoxy moiety of a polyethylene glycol derived excipient. Such moieties can be prone to air oxidation by extraction of a hydrogen atom by a reactive oxygen species having an oxygen-based radical. Not wishing to be bound by theory, extraction of a hydrogen atom from the air oxidizable excipient due to its interaction with an reactive oxygen species produced, for example by, a Fenton-type reaction elicits a radical chain reaction to generate destabilizing excipient degradants that adversely affects efficacy. This loss of efficacy is not believe to be a consequence of active pharmaceutical ingredient degradation or a drop in pH, since the former is usually not observable and the latter does not always correlate or may not be concomitant with decreased biological activity. Rather, it is postulated that loss of efficacy of the composition or formulation is a more direct consequence of air oxidation of an air oxidizable excipient, which comprises the composition or formulation.

Non-limiting examples of surface-active agents that are potentially air-oxidizable excipients have one or more polyoxyethylene chains. Such agents include polyethylene glycols (PEGs), for example those of average molecular weight from about 100 to about 20,000, typically about 200 to about 10,000 or about 300 to about 6000. Suitable PEGs illustratively include PEG 2000, having an average molecular weight of 1800 to 2200, PEG 3000, having an average molecular weight of 2700 to 3300, PEG 3350, having an average molecular weight of 3000 to 3700, PEG 4000, having an average molecular weight of 3000 to 4800, and PEG 4600, having an average molecular weight of 4400 to 4800. Other agents include poloxamers (polyoxyethylene-polyoxypropylene copolymers) such as poloxamers 124, 188, 237, 338 and 407. Other agents further include surfactants having a hydrophobic alkyl or acyl group, typically of about 8 to about 18 carbon atoms, and a hydrophilic polyoxyethylene chain. For a invention composition or formulation suspension, typically a flocculated suspension, surfactants used are typically nonionic surfactants, illustratively including polyoxyethylene alkyl ethers such as laureth-9, laureth-23, ceteth-10, ceteth-20, oleth-10, oleth-20, steareth-10, steareth-20 and steareth-100; polyoxyethylene castor oil, polyoxyethylene hydrogenated castor oil, polysorbates such as polysorbate 20, polysorbate 40, polysorbate 60, polysorbate 65, polysorbate 80, polysorbate 85 and polysorbate 120; and polyoxyethylene alkyl esters, for example polyoxyethylene stearates. In one embodiment an aqueous-based suspension of a F1C uses a Polysorbate as a surface-active agent. In another embodiment the Polysorbate is polysorbate 80. In some of these embodiments the F1C is androst-5-ene-3β,17β-diol. In flocculated suspension embodiments, the air-oxidizable surface-active agent together with any other surface active agent that can optionally be included are present in total and relative amounts that provide acceptable controlled flocculation properties.

Air oxidation of the air oxidizable excipient may take place prior to its blending into a composition or formulation and is usually an auto oxidative process whose rate depends considerably on the amount of peroxide initiator present. Accordingly, one embodiment for preparing an invention composition or formulation is to reduce the amount of peroxide burden or level before or soon after blending of an air oxidizable excipient is by using a commercially available air oxidizable excipient that has a low initial peroxide value (PV). Typically, for solutions and suspensions the peroxide value is the amount of hydrogen peroxide given in mequiv or μequiv per unit volume or mass that is equivalent to the amount of peroxide that has been determined for a test article. For an excipient, the peroxide value is typically given in mequiv O₂/Kg. For PV determinations, the ferrous oxidation with Xylenol orange assay is used as described in Ha, E. J. Pharm. Sci. 2002, 91:2252-2264 (hereby specifically incorporated by reference into the present application). Alternatively, an iodimetric technique may be used to quantify peroxide content, an example of which is described in Hamburger R., et al. Pharm. Acta Helv. 1975, 50:10-17 and Azaz, E. Analyst 1973, 98:663 (hereby specifically incorporated by reference into the present application). Another method uses a coupled oxidation to NADPH as described in Ding, S J. Pharm. Biomed. Anal. 1993, 11:95-101 (hereby specifically incorporated by reference into the present application).

In one embodiment the invention provides a method for preparing an efficacy stabilized composition or formulation that comprises (1) determining peroxide values for Polysorbate 80 lots obtained from commercial vendors; (2) selecting a lot of Polysorbate 80 for use in a invention composition or formulation that has a PV in a PV range of about 20 mequiv O₂/Kg or less, 10 mequiv O₂/Kg or less or 2.0 mequiv O₂/Kg or less or has a PV equal to or lower than is present in Polysorbate 80 obtained from Croda Health Care USA (Super Refined™ Polysorbate 80) a commercial source of this excipient.

In another embodiment, high vacuum is applied to an air oxidizable excipient either alone or in the presence of other non-volatile excipients. Removal of peroxides by vacuum from polyethylene glycols is described in Kumar, V; Kalonia, D S AAPS Pharm. Sci. Tech. 2006, 28: 7-62 (hereby specifically incorporated by reference into the present application). Typically, a vacuum of typically about 0.1 mm Hg is used. Vacuum methods to remove peroxides is effective only to the extent they are able to remove volatile components. Thus, a residual peroxide content may persist due to the presence of non-volatile peroxides. Methods to remove peroxides and other air oxidation degradants in aqueous solution of polyethylene glycols are also given in Rav, W J, Jr.; Puvanthingal, J M Anal. Biochem. 1985, 146: 307-12 (hereby specifically incorporated by reference into the present application).

In one embodiment a composition or formulation comprises a F1C and an air oxidizable excipient wherein the freshly prepared composition or formulation has a PV in a PV range of 200 μequiv H₂O₂/mL or less, 100 μequiv H₂O₂/mL or less, 50 μequiv H₂O₂/mL or less or, preferably, between about 10-20 μequiv H₂O₂/mL or less. In another embodiment the formulation is a suspension, the air oxidizable excipient is Polysorbate 80 and the F1C is androst-5-ene-3β,17β-diol.

Typically, a shelf life of 6 months to three years, typically 6 months to 2 yrs at ambient or refrigerator storage conditions is desired. A candidate composition or formulation may be evaluated for its ability to retain efficacy by (1) determining PV of the composition or formulation when freshly prepared, (2) if in acceptable PV range, heat stress the material in a stress temperature range at about 40° C. to 60 C under conditions of oxygen depletion for a stress period in a range between about 2 weeks to 3 months; (3) re-determining PV. Exact conditions for stress testing will be dependent on storage conditions and contemplated shelf life for the material, although standard testing conditions such as 60° C. and-or 40° C. with 75% relative humidity are usually suitable. In one embodiment temperatures for stress testing are chosen to provide a maximum excursion of PV during a stress period between about 2 weeks to one month. In one embodiment compositions or formulation are selected that do not reach or exceed a PV of 900 μequiv H₂O₂/mL after heating for 40° C. for 4 weeks. In another embodiment compositions or formulation are selected that do not reach or exceed a PV of 500 μequiv H₂O₂/mL after heating for 40° C. for 4 weeks. In yet other embodiment compositions or formulations are selected that do not reach or exceed a PV of 200 μequiv H₂O₂/mL or 100 μequiv H₂O₂/mL after heating for 40° C. for 4 weeks.

Typically, stress testing may be done in the absence of a metal chelator, anti-oxidant or other free radical inhibitor excipients, since these components could confound some methods of peroxide value determinations. Alternatively, degradants from degradation of an air oxidizable excipient by air oxidation may be measured and are particularly useful when free radical inhibitors or other interfering excipients that prevent accurate PV determinations are present in the composition or formulation Indices of degradants include specific aldehyde content (e.g. of acetaldehyde or formaldehyde), total aldehyde content, specific carboxylic acid content (due to subsequent oxidation of an aldehydes to a carboxylic acid including acetic or formic acids) or total carboxylic acid content. Alternatively, pH of a minimally buffered aqueous based formulation may be monitored. Aldehyde content may be evaluated spectrophotometrically after conversion to corresponding UV-vis or fluorescence detectable hydrazones. Carboxylic acid concentrations may be evaluated after conversion to corresponding fluorogenic esters as described in Khossravi, M, et al. Pharm. Res. 2002, 19: 634-9 for Polysorbate 20 (hereby specifically incorporated by reference into the present application). Typically, a invention composition or formulation with an initial PV of 200 μequiv H₂O₂/mL or less or 100 μequiv H₂O₂/mL or less will retain sufficient efficacy on storage for up to 6 months at ambient or standard refrigerated temperatures (e.g. between about +10 to −20° C.) to be suitable for treatment of a condition, although higher PV values may still allow for sufficient retention of efficacy on storage, particularly if stricter depletion of dissolved oxygen and depletion of oxygen in internal atmosphere of the container system is employed or if lower heavy metal content in the is achieved. For longer shelf life (6 months-2 years) an acceptable PV, for an air oxidizable excipient for use in preparing a F1C suspension formulation, of less than 100 μequiv H₂O₂/mL may be required with the upper limit depending on the desired extended shelf life and results from evaluating peroxide, aldehyde or carboxylic acid content during stress testing of the suspension formulation containing the air oxidizable excipient.

If stress testing indicates an unsatisfactory formulation for the required shelf life, then (1) purification of the air oxidizable excipients to a lower its PV value (2) stricter depletion of dissolved oxygen in the formulation (2) depletion of oxygen in the internal atmosphere of the container system in which the formulation is stored (3) reduction of heavy metal content in the formulation or (4) a combination of two or more of activities (1)-(3) may be required to obtain a formulation with sufficient efficacy stabilization. Mitigation or reduction of heavy metal content in the formulation may be achieved by addition of a sufficient amount of heavy metal chelator agent to the formulation to sequester heavy metal expected to be present or by limiting introduction of heavy metal into the formulation by API and excipient purification to remove heavy metal or by using manufacturing vessels and storage container systems less likely to leach out heavy metal during preparation or storage of the formulation. For example, electroplating or pacification of stainless steel in equipment to come in contact with the API, excipient or formulation may be used to limit heavy metal contamination. Additionally, specialized glass containers, septa or closures used in packaging systems for storage may be employed to limit leaching from these components.

Rate of increase in formaldehyde content in a composition or formulation is also diagnostic for composition or formulation with respect to efficacy retention during the contemplated shelf life of the material. Thus, a rate of aldehyde formation from a base level, which is the total formaldehyde content initially introduced from the blended excipients and is typically in an aldehyde range of between about 0.2 μM to 20 μM, to an aldehyde content of 100 μM or above, 200 μM or above or 400 μM or above during a stress period of e.g. 1, 2, 4 or 6 weeks of storage at 40° C. is typically indicative of a unsuitable formulation or composition. Rate of formaldehyde increase may provide an alternative method to the monitoring of PV changes for estimating shelf life, since peroxide content may fall from a maximum level achieved during stress testing, while formaldehyde content continues to rise.

A drop in pH in a solution or suspension dosage form may be used as a preliminary evaluation for measuring air oxidizable excipient degradation and a possible evaluation for its potential to lose efficacy. However, this method is not considered as reliable as the aforementioned procedures, since pH may drop for reasons unrelated to air oxidation of an air oxidizable excipient (e.g., hydrolysis of an ester bond contained within the excipient or other chemical reaction) or an increase in H⁺ production may be masked by buffer excipients. Also, it must be kept in mind that pH stabilization does not strictly correlate with efficacy stabilization, since the former may be had without the latter.

Typically, a formulation or composition is further evaluated for suitability when its PV does not increase by more than 400 mequiv H₂O₂/mL during heat stress testing under conditions of oxygen depletion. In one embodiment, formulations or compositions whose change in peroxide value is in a PV delta range of 200 mequiv H₂O₂/mL or less are selected for further evaluation. Metal chelator agents, antioxidants, etc., are also evaluated for their ability to further extend the shelf life of the drug product by minimizing PV excursion or aldehyde formation.

For administration of an aqueous-based parenteral dosage form, a sterilized drug product is required for human use. A solution composition or formulation dosage form may be sterilized by passage through a microbe-retaining filter or by heat sterilization whereas a suspension dosage from requires sterilization by input of energy, which may promote degradation. One method for sterilization by heating that minimizes oxygen exposure uses freshly obtained water for injection, which has been oxygen-depleted due to the distillation process, as the diluent when preparing the solution or suspension. The suspension or solution is then heated in a sterilization chamber or vessel (“hot sterilization” method) whose headspace is optionally replaced with an inert atmosphere before heating. Typically the sterilization chamber is fitted with a pressure relief valve that allows for passage of vapor to the external atmosphere, but which does not allow for ingress of air, and optionally further contains a valve for introduction of an inert gas (e.g. Nitrogen or Argon).

The solution or suspension is typically heated at about 121° C. or the temperature of steam compressed at 115 psi for a sterilization time between about 15 min. to 45 min, the sterilized solution or suspension is allowed to cool and pressure is equalized optionally by introduction of an inert gas either during and-or after cooling. Higher sterilization temperatures may be used if the active pharmaceutical ingredient in the solution or suspension has adequate heat stability. Lower temperatures may be used after validation. Sterilization procedures are discussed in FDA guidance to industry “Sterile drug products produced by aseptic processing” accessible at http://www.fda.gov/cber/gdlns/steraseptic.pdf (hereby specifically incorporated by reference into the present application). In one embodiment, an aqueous-based suspension comprising an F1C is hot sterilization by dispensing the suspension into individual vials, which are then sealed. Ultrasound vibration is sometimes required post sterilization if the suspension cakes during sterilization so as to give a resuspendable formulation. Ultrasound is provided at an energy and duration to effect disintegration of the cake while substantially retaining the original volume mean diameter and distribution of the suspension particles in the re-suspension. In another embodiment the bulk suspension comprising the F1C is hot sterilized which avoids caking due to continuous agitation of the suspension by mechanical stirring. The sterilized suspension is then dispensed under aseptic condition into vials to be sealed.

Alternatively, a solid active pharmaceutical ingredient or an active pharmaceutical ingredient in a blend of solid excipients may be sterilized by ionizing radiation (“cold sterilization” method) and a sterile liquid diluent or a blend of excipients dissolved in the diluent is then added to the solids so sterilized under sterile conditions. Typically, conditions employed for cold sterilization use about 25-30 kGy. After sterilization, peroxide values and aldehyde content of the parenteral dosage form may be determined before and after heat stress testing to determine if excipient degradants expected from air oxidation of an air oxidizable excipient have formed during sterilization or if new degradants have formed from nominally non-oxidizable excipient such that this excipient now act as if it were an air oxidizable excipient (i.e. a nominally non-oxidizable excipient has undergone a heat induced or radiation induced event to form a potentially destabilizing excipient degradant that otherwise would not be formed or would less likely formed during formulation storage).

Suspending agents used in suspension compositions and formulations include by way of illustration and not limitation polyvinylpyrrolidone compounds and polyethylene glycols. A Polyethylene glycol will typically have a molecular weight from about 300 to about 6000, e.g. polyethylene glycol 3350 and polyethylene glycol 4000. Polyvinylpyrrolidone (PVP) compounds will typically have a molecular weight from about 7000 to about 54000, for instance PVP K12, K17, K25 and K30. Other suspending agents are for instance cellulose derivatives such as methylcellulose, carboxymethylcellulose, hydroxyethylcellulose and hydroxypropyl-methylcellulose, gelatin and gums such as acacia. Typically, suspending agents are present in a suspension in a suspending agent range between about 0.1 to 20% w/v depending on the viscosity of the suspension in the absence of the suspending agent.

Wetting agents used in lyophilized suspension compositions and formulations, if a suspending or flocculating agent that is present does not already serve this purpose, include by way of illustration and not limitation a phospholipids/polyethylene glycol combination in a wetting agent ratio range between about 1:1 to 1:10, typically in a range between about 1:1 to 1:5, more typically between about 1:1 to 1:3.0. Suitable phospholipids for use as a wetting agent in combination with a polyethylene glycol by way of example and not limitation include mixtures of phosphatidyl choline, phosphatidyl ethanolamine, N-acylphosphatidyl ethanolamine, or phosphatidyl inositol. Further guidance in the use of wetting agents used in reconstitution of lyophilized dosage forms to give a suspension and lyophilization conditions to give the lyophilized solid to be reconstituted are given in Geller, et al. U.S. Pat. No. 5,002,940 (hereby specifically incorporated by reference into the present application). Other wetting agents that are used in suspensions are non-ionic surfactants as disclosed elsewhere and include the polyoxyethylene-sorbitan-fatty acid esters. A non-ionic surfactant serving as a wetting agent excipient in an invention composition or formulation suspension is typically present in a non-ionic surfactant range between about 0.0007% to about 3% w/v, with about 0.017 to about 0.5% w/v preferred. The minimum amount of a non-ionic surfactant such as Polysorbate 80 that may be used in an aqueous-based suspension formulation of an F1C may be estimated from the known or determined critical micelle concentration (CMC) for the diluent and non-ionic surfactant to be used. Adjustments may then be made from theoretical considerations for the presence of API and other excipients or the CMC of the supernatant of the suspension formulation may be determined from surface tension measurements using methods described in Birdi, K. S. Handbook of Surface and Colloid Chemistry, CRC Press, Boca Raton, Fla., 1997; Hiemenz, P. C. Principles of Colloid and Surface Chemistry, Marcel Dekker, N.Y. 1997. In one embodiment, preferred amounts of polysorbate 80 is present in an aqueous-based suspension formulation additionally comprising androst-5-ene-3β,17β-diol and one or more other excipients are about 0.5%, about 0.06% or about 0.016% w/v.

Compositions and formulations of the present invention may also include tonicity-adjusting agents. Suitable tonicity adjusting agents are for instance sodium chloride, sodium sulfate, dextrose, mannitol and glycerol, typically mannitol or dextrose. The effective amount of a tonicity adjusting agent will depend on the amount required to adjust an invention composition or formulation so that it is isotonic with blood

Buffers agents used include for example those derived from acetic, aconitic, citric, glutaric, lactic, malic, succinic, phosphate and carbonic acids, as known in the art. Example of buffering agents commonly used in parenteral formulations and of their usual concentrations can be found in Pharmaceutical Dosage Form: Parenteral Medications, Volume 1, 2.sup.nd Edition, Chapter 5, p. 194, De Luca and Boylan, “Formulation of Small Volume Parenterals”, Table 5: Commonly used additives in Parenteral Products (hereby specifically incorporated by reference into the present application). In one embodiment the buffering agent is phosphate or citrate buffer present in a buffering agent range between about 10-100 mM to provide a suspension or solution at an initial pH in a pH range between about 4-9, typically between about 5-8. Typically, the solution or suspension will have an osmolality in an osmolality range, typically about 286 Osmol/kg or between 229 to 342 Osmol/kg with pH in a pH range of 4.5-7.0.

For parenteral dosage form, an anti-microbial preservative is used if no other excipient that is used serves this purpose. Suitable preservatives include by way of example and not limitation phenol, resorcinol, chlorobutanol, benzylalcohol, alkyl esters of para-hydroxybenzoic acid such as methyl, ethyl, propyl, butyl and hexyl (generically referred to as parabens), benzalkonium chloride and cetylpyridinium chloride. In one embodiment an aqueous flocculated suspension of a F1C uses an edetate such as a pharmaceutically acceptable salt of EDTA as the metal chelator present in a metal chelator range such that this excipient also serves in whole or in part as the preservative. Typically, an anti-microbial preservative is present in a preservative range between about 0.001% to 1.0% w/v, typically between about 0.1 to 0.4%, more typically about 0.02% or between 0.16 to 0.24 mg/mL.

Unless otherwise stated or implied by context, expressions of a percentage of a liquid excipient in an invention composition or formulation mean the excipients percent by volume (v/v). Furthermore, expressions of an amount of a solid excipient by ratio in an invention composition or formulation means the excipients weight or volume relative to the active pharmaceutical ingredient or to the total volume of the suspension, unless otherwise stated or implied by context. Thus, 20% PEG 300 means 20% v/v PEG 300 is present in an invention composition or formulation. The amount of an excipient indicated in invention compositions is not affected by the form used, e.g., NF or USP grade solvent or excipient with the exception of an air oxidizable excipient. Thus, a non-oxidizable excipient with a grade of NF in an invention composition can be replaced with a USP counterpart, provided that other limitations stated for an invention composition or formulation are not exceeded.

Dosing Protocols or Methods.

Continuous daily dosing with the invention formulations will generally require a single dose that is administered at one or two sites once per day for about 3-7 days, usually for 4-6 days or once per day for 5 consecutive days. Treatment of a human or non-human primate after a known or potential radiation exposure will usually comprise (a) a single relatively large dose, e.g., about 400 mg or about 800 mg of androst-5-ene-3β,17β-diol in humans or (b) a course of treatment that lasts several days, e.g., intramuscular dosing once per day for 4, 5 or 6 days with a lower dose of about 100 mg or about 200 mg of androst-5-ene-3β,17β-diol in humans. A single course of androst-5-ene-3β,17β-diol (present as particles of about 5-10 μm in average particle size) treatment for 5 consecutive days at 100 mg/day or 200 mg/day by intramuscular injection of an invention suspension formulation for acute radiation exposure in adult humans is believed to be sufficient. Pediatric androst-5-ene-3β,17β-diol dosages for acute radiation exposure may be lower at about 50 mg/day or 20 mg/day for 4, 5 or 6 consecutive days. The same or similar dosing protocols can be used to treat patients that are susceptible to developing infections, e.g., in patients admitted to an intensive care unit or step down units after discharge from an intensive care unit. Such patients can have immune suppression conditions or have experienced trauma that can impair immune function, e.g., stroke, hemorrhage, bone fracture, thermal burns or other acute injuries.

Treatment of chronic conditions will typically use intermittent dosing, e.g., once daily for 3, 4 or 5 days followed by no dosing for about 2-16 weeks and another round of daily dosing for 3, 4 or 5 days with another period of no dosing for about 2-16 weeks. This treatment regimen can be maintained indefinitely as long as the clinical condition persists or the treatment continues to be indicated as useful for the patient.

In treating the pathological conditions disclosed herein, one can intermittently administer an invention composition or formulation to a subject suffering from or susceptible to a condition disclosed herein such as radiation exposure or another condition.

Intermittent dosing embodiments include administration of a invention composition or formulation parenterally and are as follows: (1) daily dosing for about 3 to about 190 days (e.g., about 3 to about 20 days), (2) no dosing of the composition or formulation for about 4 to about 190 consecutive days (e.g., about 4 to about 20 days), (3) daily dosing for about 3 to about 190 days (e.g., about 3 to about 20 days), and (4) optionally repeating the dosing protocol 1, 2, 3, 4, 5, 6, 10, 15, 20, 30 or more times. Often, the dosing of steps (1) and (3) will be maintained for about 3-15 consecutive days, usually about 3, 4, 5 or 6 consecutive days. In general, steps (1)-(3) of the dosing protocol recited above, will be repeated at least one time, typically at least 2, 3, 4, 5 or 6 times. For conditions that tend to remain chronic, the intermittent dosing protocol is typically maintained over a relatively long time period, e.g., for at least about 6 months to about 5 or more years.

One aspect of invention intermittent dosing is monitoring the subject's response to a particular dosing regimen or schedule, e.g., to any intermittent administration method disclosed herein. For example, while dosing a subject who has potentially been exposed to radiation one can measure the subject's response, e.g., amelioration of one or more symptoms or a change in infections or bleeding that is associated with exposure of a subject to radiation. An aspect of the subject's response to a composition or formulation of a formula 1 compound(s) is that the subject may show a measurable response within a short time, usually about 3-10 days, which allows straightforward tracking of the subject's response, e.g., by monitoring peripheral white blood cells (“PBMC”) or by measuring a white blood cell population(s) or expression of a cytokine or interleukin by e.g., white blood cells or a subset(s) thereof. One may monitor one or more immune cell subsets, e.g., NK, LAK, dendritic cells or cells that mediate ADCC immune responses, during and after intermittent dosing to monitor the subject's response and to determine when further administration of the formula 1 compound is indicated. These cell subsets are monitored as described herein, e.g., by flow cytometry. For any of the treatments or methods described herein, prolonged beneficial effects or a sustained biological response by a subject may result from a single administration or a few daily administrations of the formulation compound for from intermittent treatment with the formula 1 compound.

### SPECIFIC EMBODIMENTS

Aspects of the invention and related subject matter include the following specific embodiments.

Other embodiments are as described elsewhere in the specification and the claims.

1. A pharmaceutically acceptable formulation comprising one or more active pharmaceutical ingredients of formula 1 and one or more excipients wherein at least one of the excipients is an air oxidizable excipient.

2. The formulation of embodiment 1 wherein the active pharmaceutical ingredient is androst-5-ene-3β,17β-diol.

3. The formulation of embodiment 2 wherein the formulation is a suspension.

4. The formulation of embodiment 3 wherein the particles of the suspension have a volume mean diameter of about 35 μm (Dv, 0.90).

5. The formulation of embodiment 3 wherein the insoluble hydroxy steroid is present in an active ingredient range between 90.0-110.0 mg/mL.

6. The formulation of embodiment 3 wherein the air oxidizable excipient is a surface-active agent.

7. The formulation of embodiment 6 wherein the surface-active agent is a suspending agent or a wetting agent

8. The formulation of embodiment 7 wherein the surface-active agent is a polysorbate.

9. The formulation of embodiment 8 wherein the polysorbate is Polysorbate 80.

10. The formulation of embodiment 6 wherein the peroxide value of the formulation prior to parenteral administration to a subject is in a peroxide value range between about 100-200 μequiv H₂O₂ or less.

11. The formulation of embodiment 6 wherein the peroxide value of an air oxidizable excipient is substantially the same as the PV for Polysorbate 80 obtained from Croda Health Care USA as Super Refined™ Polysorbate 80.

12. The formulation of embodiment 6 wherein the formulation additionally comprises a metal chelator agent.

13. The formulation of embodiment 12 wherein the metal chelator agent is an edetate.

14. The formulation of embodiment 13 wherein the metal chelator agent is present in a metal chelator agent range between about 0.01 % to 0.05% w/v.

15. The formulation of embodiment 6 wherein the formulation additionally comprises an antioxidant.

16. The formulation of embodiment 6 wherein the suspension additionally comprises a citrate buffered aqueous solution.

17. The formulation of embodiment 16 wherein the suspension has an osmolality of about 286 Osmol/kg.

18. The formulation of embodiment 6 wherein the formulation additionally comprises an anti-microbial preservative.

19. The formulation of embodiment 18 wherein the preservative is benzalkonium chloride.

20. The formulation of embodiment 19 wherein the preservative is present in about 0.2 mg/mL.

21. The formulation of any one of embodiments 1-20 wherein the active pharmaceutical ingredient is androst-5-ene-3β,17β-diol or a hydrate thereof

22. The formulation of Table A, Table B or Table C

23. Use of composition to prepare a medicant wherein the composition comprises a water insoluble F1C and polysorbate 80

24. The use according to embodiment 23 wherein the F1C is androst-5-ene-3β,17β-diol.

25. Use of a compound to prepare a medicament for the treatment of radiation exposure wherein the composition comprises androst-5-ene-3β,17β-diol and polysorbate 80.

1A. An efficacy stabilized pharmaceutically acceptable formulation comprising a active pharmaceutical ingredient or a pharmaceutically acceptable salt or a hydrate thereof an at least one air oxidizable excipient wherein the formulation is a suspension for parenteral administration to a subject; wherein the active pharmaceutical ingredient is androst-5-ene-3β,17β-diol, androst-5-ene-3β,7β,17β-triol or an ester, ether or hydrate thereof.

2A. The formulation of embodiment 1A wherein the air oxidizable excipient is a non-ionic surfactant.

3A. The formulation of embodiment 1A wherein the air oxidizable excipient is a suspending agent, a flocculating agent, a wetting agent or a diluent.

4A. The formulation of embodiment 2A or 3A wherein the formulation is essentially free of a destabilizing excipient degradant wherein the degradant is derived from air oxidation of the air oxidizable excipient.

5A. The formulation of embodiment 4A wherein the destabilizing excipient degradant is a reactive oxygen species or an aldehyde

6A. The formulation of embodiment 1A wherein the active pharmaceutical ingredient is androst-5-ene-3β,17β-diol.

7A. The formulation of embodiment 6A wherein the compound is androst-5-ene-3β,17β-diol or a hydrate thereof.

8A. The formulation of embodiment 1A wherein the formulation is a suspension for intramuscular or subcutaneous administration.

9A. The formulation of embodiment 8A wherein the air oxidizable excipient is a surface-active agent.

10A. The formulation of embodiment 8A wherein the air oxidizable excipient is a non-ionic surfactant.

11A. The formulation of embodiment 10A wherein the non-ionic surfactant contains an unsaturated fatty acid ester or a polyethylene glycol ether.

12A. The formulation of claim 10A wherein the non-ionic surfactant is Polysorbate 80 or Polysorbate 40.

13A. The formulation of embodiment 3A wherein the air oxidizable excipient is a polyethylene glycol.

14A. The formulation of embodiment 13A wherein the polyethylene glycol has a molecular weight in the molecular weight range of about 300-1000 AMU.

15A. The formulation of embodiment 12A additionally comprising at least one free radical inhibitor agent.

16A. The composition of embodiment 15A wherein the free radical inhibitor agent is an antioxidant.

17A. The formulation of embodiment 12A additionally comprising a metal chelator agent wherein the metal chelator agent is an acid or pharmaceutically acceptable salt of ethylenediaminetetraacetic acid (EDTA), ethyleneglycoltetraacetic acid (EGTA), diethylene-triaminepentaacetate (DTPA), hydroxyethylethylene-diaminetriacetic acid (HEEDTA), diaminocyclohexane-tetraacetic acid (CDTA) or 1,2-bis(2-aminophenoxy)ethane-N,N,N′,N′-tetraacetic acid (BAPTA) or a combination thereof.

18A. The formulation of any one of embodiments 6A-17A wherein the composition is essentially free of a destabilizing excipient degradant wherein the degradant is derived from air oxidation of the air oxidizable excipient.

19A. The formulation of embodiment 18A wherein the destabilizing degradant is an aldehyde.

20A. The formulation of embodiment 19A wherein the aldehyde is formaldehyde.

21A. The formulation of embodiment 18A wherein the destabilizing degradant is a reactive oxygen species.

22A. The formulation of embodiment 21A wherein the reactive oxygen species is a peroxide.

23A. The formulation of embodiment 1A wherein the formulation is a suspension wherein at least one excipient is a liquid vehicle and wherein the oxidizable excipient is a non-ionic surfactant present in a non-ionic surfactant range between about 0.1% to 1.0%.

24A. The formulation of embodiment 23A wherein the non-ionic surfactant agent is Polysorbate 80 or Polysorbate 40.

25A. The formulation of embodiment 24A wherein the liquid vehicle is a buffered aqueous solution present in a liquid vehicle range between about 2.5-1,000 mL per gram of active pharmaceutical ingredient.

26A. The formulation of embodiment 25A wherein the buffered aqueous solution is a phosphate or citrate buffer with buffering agent present in a buffering agent range between about 10-100 mM at an initial pH in a pH range between about 4-9.

27A. The formulation of embodiment 26A wherein the active pharmaceutical ingredient is androst-5-ene-3β,17β-diol or a hydrate thereof;

wherein the liquid vehicle is a mixture of sodium phosphate mono basic and sodium phosphate dibasic in water for injection;

wherein the suspension has an osmolality between 229 to 343 mOsmol/kg;

wherein the suspension has an initial pH within a pH range of about 4-7.5;

wherein the oxidizable excipient is Polysorbate 80 present in about 0.5% w/v

28A. The formulation of embodiment 27A additionally comprising a free radical inhibitor wherein the free radical inhibitor is a heavy metal chelator agent.

29A. The formulation of embodiment 28A wherein the metal chelator is an acid or pharmaceutically acceptable salt of EDTA, or a combination thereof, present in a metal chelator range between about 0.01 to 0.05% w/v.

30A. A sterile efficacy stabilized pharmaceutical formulation for a parenteral administration to a subject prepared by the process of

(1) mixing freshly prepared water for injection to a mixture comprising a F1C and a vehicle, wherein the formulation contains at least one air oxidizable excipient;

(2) replacing the headspace in a closed vessel over a formulation of claim 1;

(3) depleting oxygen dissolved in the formulation;

(3) heating the vessel at a sterilization temperature of 121 C within a sterilization time range between about 15-45 min;

wherein the formulation is a suspension and the F1C is androst-5-ene-3β,17β-diol, androst-5-ene-3β,7β,17β-triol or an ester, ether or hydrate thereof.

31A. The formulation of embodiment 30A wherein the active pharmaceutical ingredient is 3β,17β-di-hydroxy-androst-5-ene, or a hydrate thereof, additionally comprising mannitol, benzalkonium chloride, sodium phosphate monobasic, sodium phosphate dibasic, EDTA and water for injection.

32A. The formulation of embodiment 30A wherein the formulation has the specifications of Table A, Table B or Table C.

33A. The formulation of embodiment 30A wherein the formulation has the specifications of Table B.

34A. A method for treating a subject with an immune suppressive condition, an unwanted immune response, a blood disorder deficiency, radiation exposure or a symptom thereof by administering to a subject with said condition, response, deficiency or exposure a therapeutically effective amount of an efficacy stabilized formulation of any one of embodiments 30A-33A

35A. The method of embodiment 34A wherein the subject has had or may suffer from radiation exposure.

### EXAMPLES

The following examples further illustrate the invention and they are not intended to limit it in any way.

### Example 1

Determination of peroxide value by the Xylenol orange method. The peroxide values for invention compositions or formulations are given in μEq H₂O₂ (equivalent to the H₂O₂ concentration in μM). Commercial available analytical standard hydrogen peroxide solution was used to make standard working solution from about 4 μM to 90 μM after serial dilution with DI water. Fox Reagent was prepared by dissolving 49 mg ammonium iron (II) sulfate, hexahydrate, 38 mg of xylenol orange tetrasodium salt, and 9.1 g of sorbitol into 500 ml of 25 mM H₂SO₄ solution. A sample of the solution from a solution composition or formulation or the supernatant of a suspension composition or formulation (test article) were used directly. If the peroxide concentration was found to extend beyond the linear range of the peroxide calibration curve, appropriate dilutions with water of the effected samples were carried out. For construction of the calibration curve and analysis of test artricle, 1900 μL Fox reagent is pipeted a 4 mL vial to which a 100 μL peroxide standard solution or test article is added and is allowed to set for 20 min for color development. Each sample was run in quadruplets. Absorbance measurements at 570 nm were performed in 96-well plate format using 300 μL of the developed sample to the designated well. Read absorbance of 96-well plate. The average absorbance of a peroxide standard was plotted against its peroxide concentration. The resulting linear equation derived from the plot was used to calculate peroxide levels of test article.

### Example 2

Determination of formaldehyde concentration. Formaldehyde standard solutions were prepared from commercial analytical grade formaldehyde to make standard working solution from 10 μM to 500 μM after serial dilution with DI water. A 10 mM 2,4-Dinitrophenylhydrazine (DNPH) solution was prepared from commercial analytical grade DNPH and 1N HCl (aq). To a 2 mL HPLC sampling vial was added 400 μL of a HCHO standard solution and 200 μL 10 mM DNPH solution. To prepare blank solutions, distilled water replaced the HCHO standard solution. A 400 μL sample of the solution from a solution composition or formulation or the supernatant of a suspension composition or formulation (test article) were used directly for analysis. The samples were then allowed to set for at least for 2 hours before HPLC analysis. HPLC analysis used an Agilent Eclipse XDB-C18, 3.5 μm, 4.6×150 mm column eluting with 0.1% TFA in water (A) and 0.1% TFA in acetonitrile (B) isocratic with 50% B at a flow rate of 1.0 mL/min with an injection volume of 20 μL. Detection used 355 nm with reference signal off. The area of the peak corresponding to 2,4-dinitrophenylhydrazone of formaldehyde was plotted against the formaldehyde concentration of a standard solution. The resulting linear equation from the resulting plot was used to calculate formaldehyde concentration of test article.

### Example 3

Study on the effect of excipients on peroxide value in developing a suspension formulation.

The suspension formulation of Table A was investigated to determine effect on peroxide number, when using androst-5-ene-3β,17β-diol as the active pharmaceutical ingredient and high purity Polysorbate 80 as the wetting agent, by various excipients through systematic removal of one excipient.

TABLE A

| androst-5-ene-3β,17β-diol (AED) | 100 mg/mL in WFI |
|---|---|
| Polysorbate 80¹ | 0.5% |
| Na₂HPO₄ | 0.012% |
| NaH₂PO₄ | 0.08% |
| Benzalkonium chloride 50% | 0.04% |
| Mannitol | 4% |

¹Super Refined ™ Polysorbate 80 from Croda Health Care, USA with a stated maximum PV of 2.0 mequiv O₂/Kg and maximum formaldehyde content of 10 ppm.

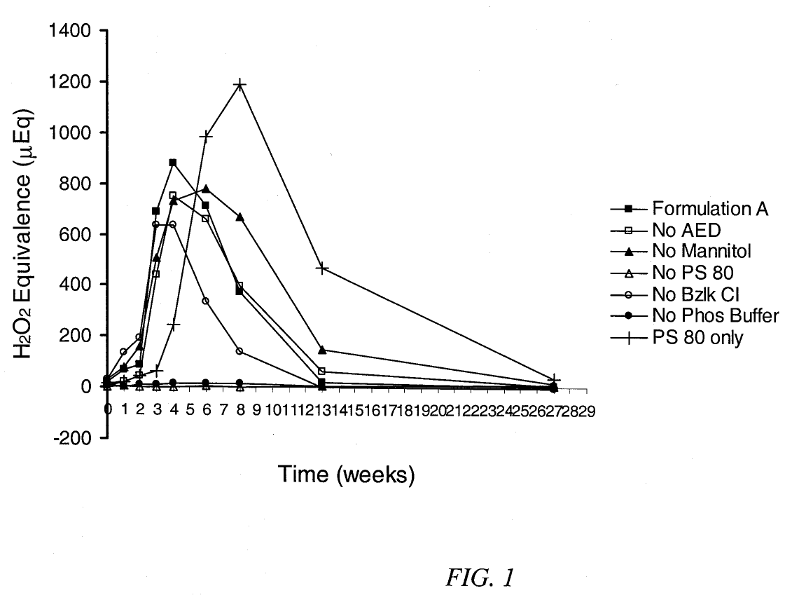

Effects on PV form heat stressing the suspension formulation of Table A at 40° C. in the absence of one excipient are given in FIG. 1. “Formulation A” is the formulated suspension according to Table A with 3β,17β-di-hydroxy-androst-5-ene as the active pharmaceutical ingredient with all excipients included. “No-AED” is the formulation of Table A without the active pharmaceutical ingredient absent. “No PS80” is the formulated suspension according to Table A without the wetting agent. “No mannitol” is the formulated suspension according to Table A without the tonicity agent. “No Phos Buffer” is the formulation according to Table A absent the buffering agents. “No Benzalk Cl” is the formulation of Table A absent the anti-microbial preservative agent. Stress testing was conducted under an oxygen-depletion method wherein the headspace in the vial is replaced with nitrogen.

The study indicates that a phosphate-based buffer system may be problematic in developing an efficacy-stabilized formulation when used in combination with the other excipients listed.

### Example 4

Study on the effect of excipients on aldehyde formation in developing a suspension formulation.

The suspension formulation of Table A was investigated to determine effect on formaldehyde formation, when using androst-5-ene-3β,17β-diol (AED) as the active pharmaceutical ingredient and high purity Polysorbate 80 as the wetting agent, by various excipients through systematic removal of one excipient.

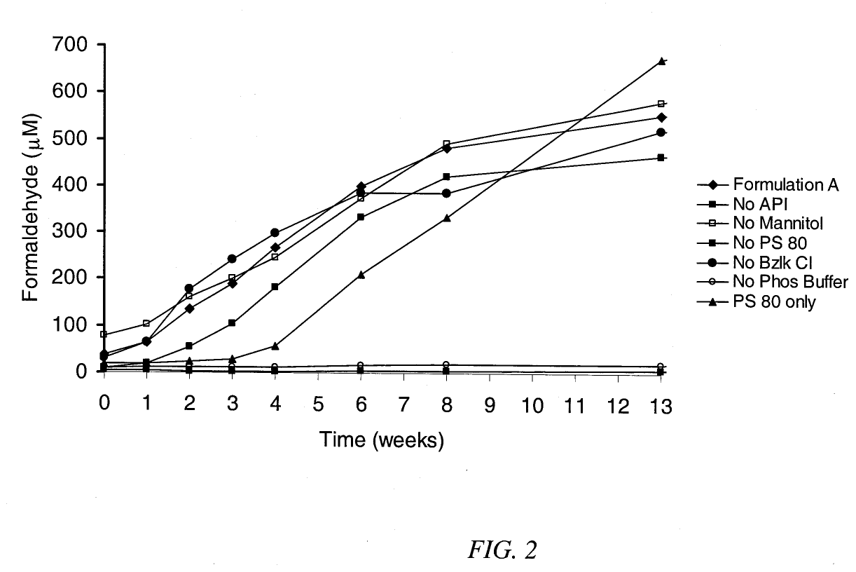

Effects on formaldehyde formation form heat stressing the suspension formulation of Table A at 40° C. in the absence of one excipient are given in FIG. 2. Stress testing was conducted under an oxygen-depletion method wherein the headspace in the vial is replaced with nitrogen.

The study indicates that a phosphate-based buffer system may be problematic in developing an efficacy-stabilized formulation when used in combination with the other excipients listed. The study also shows that following formaldehyde content would be useful in examining similar formulations for predicting shelf life.

### Example 5

Study to determine effects of environment, metal chelator excipient and sterilization conditions on aldehyde formation in formulation development.

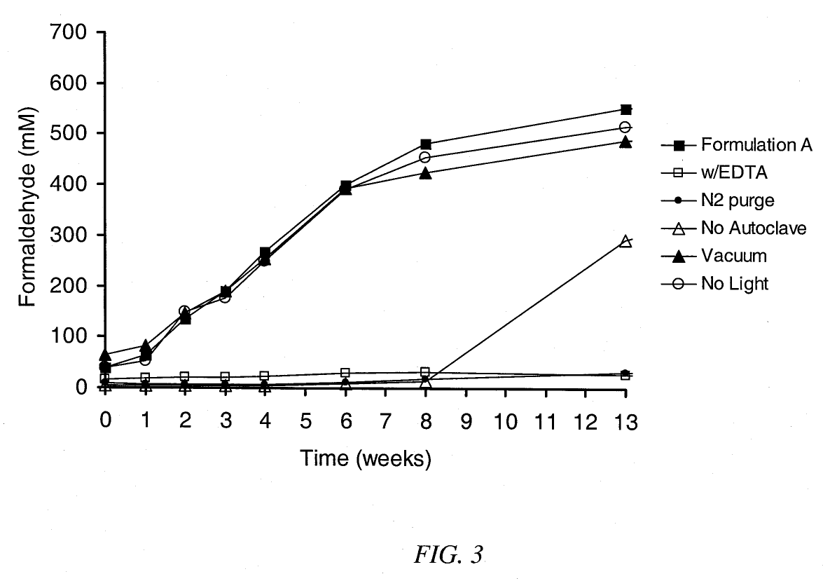

The suspension formulation of Table A was investigated to determine effect on formaldehyde formation by exposure to light, presence of an edetate, sterilization without using an oxygen-depletion method and using the additional oxygen-depleting method of nitrogen sparging (in combination with replacing the head space in the vial with nitrogen) during stress testing. Results are presented in FIG. 3.

Results indicate that typical a “hot sterilization” method that does not employ a method of oxygen depletion results in significant formaldehyde production, whereas employing an oxygen depletion method that removes dissolved oxygen prior to sterilization (e.g. by sparging) is predicted to be beneficial. The line in FIG. 3 identified “w/EDTA” represents Formulation A with the addition of 0.05% w/v disodium EDTA dihydrate and indicates that an edetate will prevent the formation of formaldehyde presumably through inhibiting auto-oxidation of Polysorbate 80 mediated by a heavy metal ion.

### Example 6

Effect of formulation stabilization on efficacy based upon Platelet Count after non-lethal radiation

Example formulations studied are given in Tables B and C

TABLE B
(Stabilized Formulation)

| androst-5-ene-3β,17β-diol | 100 mg/mL in WFI |
|---|---|
| Polysorbate 80 | 0.5% |
| Na₂HPO₄ | 0.012% |
| NaH₂PO₄ | 0.08% |
| Benzalkonium chloride 50% | 0.04% |
| Mannitol | 4% |
| EDTA di-sodium di-hydrate | 0.05% |

TABLE C
(Formulation Lacking Stabilization)

| androst-5-ene-3β,17β-diol | 100 mg/mL in WFI |
|---|---|
| Polysorbate 80 | 0.5% |
| Na₂HPO₄ | 0.012% |
| NaH₂PO₄ | 0.08% |
| Benzalkonium chloride 50% | 0.02% |
| Mannitol | 0-10% |
| EDTA di-sodium di-hydrate | 0.0% |

In formulations of Tables A, B and C, androst-5-ene-3β,17-diol monohydrate was blended into the suspension to achieve a 100 mg/mL suspension of androst-5-ene-3β,17β-diol (API). Initial pH values of the suspension formulations in Table B and C were pH 6.

Study Protocol: Non-Lethal radiation of non-human primates. The test articles of suspension formulations of Table B and C and vehicle/control formulations in the amount of 0.15 ml/kg was administered once daily by intramuscular (IM) injection for 5 days. The first dose was administered 1-3 hours after whole-body irradiation with 440 cGy delivered in the following manner. Subjects were 5 Macaca mulatta (Rhesus monkey) weighing between 4-8 kg and aged between 4-7 years at onset of treatment. Vehicle control formulation was formulation C absent the active pharmaceutical ingredient. Immediately prior to drawing a suspension test article into a syringe, the test article formulations were vortexed to uniformly distribute sediment in test article. Once drawn into a syringe, the test articles were administered within 10 minutes. Just prior to an injection, the syringe containing the test article was rotated end-over-end to uniformly disperse the test article suspension.

Animal management was conducted as follows. Upon arrival all animals were subjected to a detailed physical examination and body weight measurement by the technical staff under the direction of the attending veterinarian. In addition, blood was collected from all animals (not food and water deprived) and assessed for basic blood chemistry. The results of the evaluations were reviewed by a veterinarian to ensure satisfactory health status. Animals were housed individually in stainless steel squeeze back cages equipped with an automatic watering system except during transportation where water bottles are provided. The animal room environment was controlled (temperature 21±3° C., humidity 30-70%, 10-15 air changes per hour, 12 hours light, 12 hours dark). Temperature and humidity were monitored continuously. Wheat and corn-based primate chow (obtained from the monkey breeding facility where these animals were bred and raised) were made available to each monkey daily. Food was withdrawn overnight prior to radiation. Commercially available drinking water (distilled water) was supplied to animals ad libitum. Housing, experiments and all other conditions were approved by an ethics committee in conformity with local regulations.

Whole body radiation was conducted as follows. Each animal received a midline treatment dose of 440 cGy. The dose rate of the ⁶⁰Co gamma source was approximately 40 cGy per minute. In order to produce homogenous dose distribution, treatment was divided into two phases. First, the animal received half of the dose by anterioposterior (AP) irradiation. The second half of the dose was delivered by posteroanterior (PA) irradiation. The radiation dose was calibrated using an acrylic phantom placed in the same experimental set up that was used for irradiation of the subjects.

Clinical pathology was conducted as follows. Laboratory hematology investigations were performed on all animals three (3) times during the pre-treatment period and daily during the treatment period on Days 2, 5, 8, 10-27, 30, 33, 36 and 40. On the days that animals are to receive test articles, blood samples for hematology were taken right before the treatments. Blood samples of about 1 mL were collected from the femoral vein or from any appropriate vessel by venipuncture for hematological analysis. Animals were not deprived of food or water prior to blood collections.

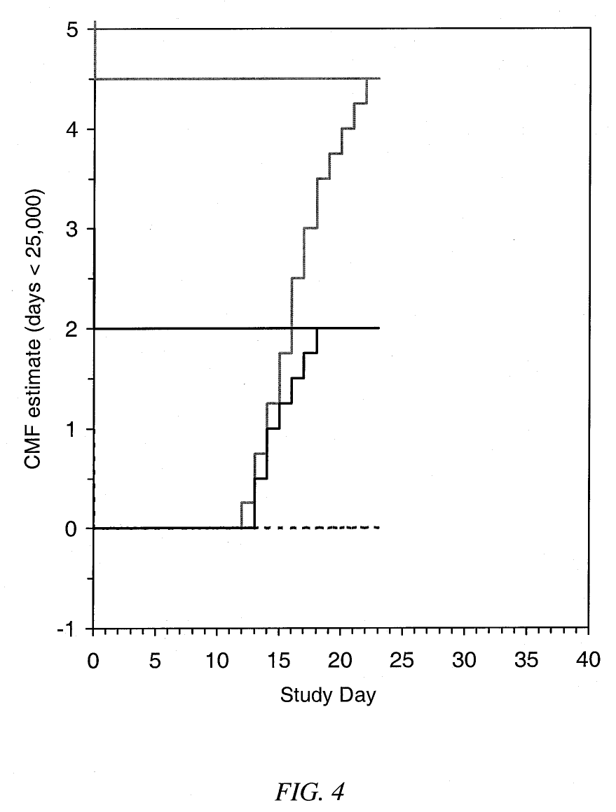

Differences in platelet counts after whole body radiation between vehicle and treatment with formulations containing API are shown in the Cumulative Mean Function (CMF) plot of FIG. 4. The bottom stepped dashed line represents the mean days platelet levels fell below 25,000 when radiation exposure was treated with the formulation of Table B (stabilized). The middle stepped dark line represents treatment with the formulation of Table C (lacking stabilization). The top stepped grey line is for vehicle control represented by Table C but absent the active pharmaceutical ingredient.

### Example 7

Effect of formulation on efficacy based upon Neutrophil Count after non-lethal radiation. Study Protocol: Non-Lethal radiation of non-human primates.

Primates used in this study were purpose bred rhesus monkeys (Macaca Mulatta), weighing 2.5 to 4.0 Kg each and aged 2 to 3 years. 5-Androstene-3β,17β-diol (5-AED), prepared as a 100 mg/mL aqueous suspension using 7.4 mM sodium phosphate buffer, pH 6.0, containing 0.5% polysorbate 80, 0.02% benzalkonium chloride and 4.8% mannitol and its vehicle, consisting of the identical formulation without 5-AED, was administered intramuscularly (i.m.) at a dose of 15 mg/kg/d (150 μL/Kg/d), for 5 consecutive days. For the first study, a total of 14 monkeys were randomly selected to receive either 5-AED (2 females, 4 males) as a stabilized formulation or vehicle (4 females, 4 males). The drug and placebo carrier were administered at exactly two hours after total body irradiation (TBI) and then every 24 hr thereafter for a total of 5 consecutive days. 5-AED and vehicle were administered by deep intramuscular injections into alternating left and right m. vastus lateralis. In the second study, 10 animals (3 males and 2 females in each group) received either 15 mg/Kg/d (150 μL/Kg/d), 5-AED in a stabilized formulation or vehicle for 5 consecutive days, but were not subjected to TBI.

Animal management was conducted as follows: The monkeys were housed individually in stainless steel cages in rooms equipped with reverse-filtered air barrier, provided with normal daylight rhythm, and conditioned to 20° C. with a relative humidity of 70%. Animals were fed ad libitum with commercial primate chow, fresh fruits, and acidified drinking water. All animals were free of intestinal parasites and seronegative for Herpes B, simian T-lymphotropic viruses (STLV), simian immunodeficiency virus (SIV), Ebola and Hepatitis B virus. Housing, experiments and all other conditions were approved by an ethics committee in conformity with local regulations. Approximately two weeks before TBI, monkeys were placed in a laminar flow cabinet and the gastrointestinal tract was selectively decontaminated by giving orally a single dose of Piperazine and Yomesan, starting at day 11 before TBI, followed by Flagyl, Madicure, and Chloroquine for 7, 5 and 10 days respectively. Subsequently treatment with oral preparations of Ciprofloxacin, Nystatin and Polymyxin B was initiated and continued all through the experiment. In addition, this regimen was supplemented with systemic antibiotics, Piperacillin and Cefuroxim, when leukocyte counts dropped below 1.0×10⁶/mL. Administration of all antibiotics was discontinued when leukocyte counts reached levels of 1.0×10⁶/mL for 3 consecutive days. Nystatin treatment was continued for another additional 10 days. During decontamination, iron supplementation, Cosmofer, was administered 5 times by deep i.m. injections by alternating the left or right upper leg. Dehydration and electrolyte disturbances were treated by appropriate fluid and electrolyte administration. Monkeys received irradiated (15 Gy γ-rays; Gammacell 40; Atomic Energy of Canada, Ottawa, Canada) whole blood transfusions, whenever platelet counts reached values below 40×10⁶/mL, or whenever hematocrits were <20%. Ten to 20 mL peripheral blood of healthy male donor monkeys was collected 1:10 in a sodium citrate solution. Donor monkeys were treated with 2.5 μg/Kg/d for 4 consecutive days with rhesus TPO, after which platelets started to increase to 10 times the normal physiological levels. The criterion of transfusion of platelets at counts <40×10⁶/mL was chosen because monkeys already develop petechiae and other hemorrhages at this level.

Whole body radiation was conducted as follows: Rhesus monkeys were irradiated with a single dose of 6 Gy TBI delivered by a 6 MV linear accelerator (Siemens). During irradiation the monkeys were anesthetized with Ketamine and placed in a perspex frame. The dose rate was 31 cGy/min and the focus-skin distance was 2 meters. The irradiation was delivered in two parts, half of the dose in anterior-posterior (AP) position, and the other half in PA position. The dose was later confirmed by means of TLD fixed on the frame, close to the monkey. In keeping with a relative biological effectiveness (RBE) of 0.85, the dose of 6 Gy is equivalent to the dose of 5 Gy 300 kV X-rays found to be the mid-lethal dose without supportive care.

Clinical pathology was conducted as follows: Bone marrow was aspirated under neurolept anesthesia using Ketalar (Apharmo, Arnhem, the Netherlands) and Domitor (Pfizer, Capelle a/d lJssel, The Netherlands). Small bone marrow aspirates for analytical purposes were taken from the shafts of the femurs or humeri using pediatric spinal needles and collected in bottles containing 2 mL HEPES buffered Hanks' balanced salt solution (HBBS) with 200 IU sodium heparin/mL (Leo Pharmaceutical Products, Weesp, the Netherlands). Low-density cells were isolated using Lymphoprep (density 1.077, Fresenius, Oslo, Norway) separation. Cells were plated in 35-mm dishes (Falcon 1008, Becton Dickinson, Leiden, The Netherlands) in 1 mL enriched Dulbecco's medium containing 0.8% methylcellulose, 5% FCS, and additives. For burst-forming units-erythroid (BFU-E), cultures were supplemented with hemin (2×10⁻⁴ mol/L), human recombinant erythropoietin (Epo; 4 U/mL; Behring, Germany) and Kit ligand (KL; 100 ng/mL; Immunex Seattle, Wash.). For granulocyte/macrophage colony-forming units (GM-CFU), cultures were supplemented with recombinant human GM-CSF (5 ng/mL; Behring), recombinant rhesus monkey IL-3 (30 ng/mL), produced in B. licheniformis and purified as described previously, and KL. Low-density cells were plated at 5×10⁴ cells per dish in duplicate. Colony counts were calculated per mL of bone marrow aspirated using the recovery of cells over the Ficoll density gradient. Colony numbers represent the mean±standard deviation of bone marrow samples of individual monkeys. Complete blood cell counts were measured daily using an ABC-vet animal blood counter (Scil, ABX diagnostics, Montpellier, France). For reticulocyte measurements, 5 μL EDTA blood was diluted in 1 mL PBS/EDTA/sodiumazide and one mL of a Thiazole Orange (TO) dilution was added, using TO at a final concentration of 0.5 μg/mL. Measurements were done using a FACSCalibur (Becton Dickinson, Leiden, The Netherlands) and 50,000 events were collected in duplicate and analyzed using the CellQuest (Becton Dickinson) software. Once weekly, a FACS analysis was done on peripheral blood (PB) and bone marrow (BM) samples on the following surface antigens: CD2, CD4 and CD8 (T-cells), CD20 (B-cells), CD11b (myelomonocytes), CD56 and CD16 (NK cells) and CD34 (immature cells) using directly labeled monoclonal antibodies (Becton Dickinson). A monoclonal antibody against human HLA-DR, which reacts with rhesus monkey RhLA-DR antigens (Becton Dickinson), was used to measure HLA-DR activated CD34+ cells. Whole blood or bone marrow was lysed in lysing solution (8.26 g ammonium chloride/1.0g potassium bicarbonate and 0.037 g EDTA per L) for 10 minutes at 4° C. After lysing, the cells were washed twice with HBBS containing 2% BSA and 0.05% (w/v) sodium azide. The cells were resuspended in 100 μL of the latter fluid containing 2% normal monkey serum to prevent non-specific binding of the monoclonal antibodies. Monoclonal antibodies were added in a volume of 2.5 to 5 μL each and incubated for 30 minutes on ice in the dark. After two washes, the cells were measured on a FACSCalibur in the presence of Propidium Iodide (Sigma Aldrich, Zwijndrecht, The Netherlands). Un-gated list mode data were collected for 10,000 events and analyzed using the CellQuest software (Becton Dickinson). Blood samples to measure serum concentrations of sodium, potassium, chloride, glucose, albumin, total protein, aspartate-amino transferase, alanine-amino transferase, alkaline phosphatase, lactate dehydrogenase (LDH), gamma-glutamyl transpeptidase, bilirubin, C reactive protein, creatinin, urea and bicarbonate are collected once a week, for retrospective analysis if indicated using an Elan Analyzer (Eppendorf Merck, Hamburg, Germany). At 2, 4, 8, 12 and 24 hours after irradiation and then every day for 42 days 150 μL of EDTA plasma, pre-irradiation baseline, was collected and stored at −80° C. At the end of the study all samples were processed for 5-AED levels, 5-AED metabolite levels in addition to more specific cytokine and hematopoietic growth factor measurements.

Summary of results are the following: In the present study, rhesus monkeys were subjected to 6 Gy TBI and treatment with a stabilized formulation of 15 mg/kg i.m. 5-AED (n=6) or vehicle (n=8) for 5 consecutive days, starting 2 hours after irradiation. TBI resulted in profound pancytopenia in all monkeys. Treatment with the 5-AED formulation reduced the period of leukopenia by 4 days. This could be attributed to accelerated neutrophil recovery (P<0.01), and was also reflected in CD11b+ cells (P<0.01), CD16+ (P<0.01) and CD56+ (P<0.05) NK cells. Recovery of reticulocytes was markedly enhanced in the 5-AED group and reached levels >0.05×10⁹/mL in peripheral blood (PB) by day 19.0±1.1, whereas HERF-418 control monkeys did not reach this level until day 25.3±5.8 (P<0.05). A prominent effect of the stabilized formulation of 5-AED was also noted for platelet recovery, since 5-AED both decreased the need for transfusions, with only 1.3±0.5 transfusion needed to maintain platelets levels of >40×10⁶/mL as opposed to 3.4±2.8 in the HERF-418-treated monkeys, as well as shortened the time to transfusion-independence by 4 days (P<0.05). Accelerated recovery of bone marrow cellularity was observed at day 22 after TBI in the 5-AED treated group 8.7±5.3×10⁶ cells/mL bone marrow aspirate versus 1.5±2.0×10⁶ cells/mL for HERF-418-treated monkeys (P<0.01). CD34+ cells in BM of 5-AED monkeys showed a 90-fold increase in comparison to HERF-418 treated monkeys as early as day 15 after TBI, which was also reflected in accelerated recovery of clonogenic progenitor cells. Treatment of non-irradiated monkeys with the stabilized formulation of 5-AED (n=5) resulted in a 3.6 fold increase from baseline levels in neutrophilic granulocytes in the peripheral blood with a maximum at day 2 after initiation of the treatment, but did not affect other hematopoietic lineages or bone marrow cellularity and progenitor cell content. Direct local or systemic toxic effects were not observed during administration of the steroid, but all 5-AED monkeys, both irradiated and non-irradiated, displayed an increase of up to 13.6% in body weight due to fluid retention in the 2^{nd} week, resulting in transient edema, which resolved without sequela. This preclinical study characterizes this stabilized formulation of 5-AED as a potent novel agent to promote stem cell reconstitution and multilineage myelopoiesis after radiation-induced bone marrow suppression, resulting in enhanced reticulocyte, neutrophil and platelet recovery. Mean, median and range of numeric variables reported herein were calculated by the Excel spreadsheet program. Standard deviations were calculated on the assumption of a normal distribution. The statistical significance of differences was calculated with the Mann-Whitney test, comparing two unpaired groups each time.

### Example 8

Combination Treatment with androst-5-ene-3β,17β-diol formulation and TPO. For direct measurements of the radioprotective effect of 5-AED, BALB/c mice were exposed to a midlethal dose of 6 Gy TBI. Two hours after TBI, mice were injected IM with 40 mg/kg 5-AED or the carrier as placebo, with or without 0.225 μg TPO or 10 μg Peg-G-CSF IP. Radioprotective effects of 5-AED on immature repopulating cell subsets were assessed by exposing BALB/c donor mice to 3 fractions of 2 Gy TBI, separated by 24 hours, and treatment with 40 mg/Kg/d 5-AED or the carrier IM, or 0.7 μg TPO IP after each fraction or a single injection of 10 μg Peg-G-CSF IP after the first fraction. Twenty four hours after the last fraction, bone marrow of donor mice was examined for immature cell content per femur using the marrow repopulating ability (MRA day 13) assay and the CFU-S day 12 after transplantation in 8 Gy irradiated mice. After 6 Gy TBI, BALB/c mice treated with 5-AED displayed an accelerated multilineage recovery with increased white blood cells (P<0.001), blood platelets (P<0.0001) and red blood cells (P<0.03), as well as increased bone marrow cellularity (P<0.0001) and elevated numbers of bone marrow colony forming cells (P<0.00001) at 14 days post-TBI in comparison to placebo-treated animals. Increasing the 5-AED dose up to 200 mg/kg did not augment this effect. Combined treatment with 5-AED and Peg-G-CSF or TPO treatment did not result in an additive effect in this setting. However, after the fractionated 3×2 Gy, a 5- and 7-fold increase in CFU-S relative to radiation controls was observed in the 5-AED and TPO groups, respectively, and a synergistic 20-fold increase in CFU-S day 12 was observed when 5-AED and TPO were used simultaneously. Consistent with earlier observations, Peg-G-CSF alone did not affect CFU-S day 12 and appeared to dampen the effect of 5-AED. MRA, expressed as GM-CFU per femur at 13 days after transplantation, was found to be increased 5- to 6-fold with 1002 colonies (range 0-5785) for 5-AED versus 174 (5-360) for radiation controls. This is in contrast to TPO, which promotes CFU-S reconstitution at the expense of the more immature MRA (Neelis et al. 1998: Blood 92, 1586). Thus, 5-AED as a single agent stimulates multilineage hematopoiesis and increases bone marrow cellularity following TBI. This effect is mediated by increased survival and/or reconstitution of immature repopulating cells in a pattern distinct from that of TPO. Consistently, 5-AED strongly synergizes with TPO at the level of immature cells from which reconstitution originates, thus revealing a novel mechanism of bone marrow protection in cytoreductive therapy.

What is claimed is:

1. A aqueous suspension formulation comprising an F1C, a pharmaceutically acceptable aqueous-based diluent and at least one pharmaceutically acceptable, air oxidizable excipient wherein the F1C is androst-5-ene-3β,17β-diol, androst-5-ene-3β,7β,17β-triol or an ester or ether derivative of either of these compound and wherein the formulation
contains less than about 1-2 ppm of dissolved oxygen or is essentially free of dissolved oxygen
or contains less than about 25-160 ppm of lead equivalent of heavy metal, less than about 1-30 ppm or essentially free of heavy metal wherein the heavy metal is one, two, three or more metals selected from the group consisting of iron, cobalt, copper, chromium, vanadium
or has an initial peroxide value of 100 μequiv H₂O₂/mL or less.

2. The suspension formulation of claim 1 wherein the active pharmaceutical ingredient is androst-5-ene-3β,17β-diol, the air oxidizable excipient is an air oxidizable surface-active agent and the heavy metal is Fe.

3. The suspension formulation of claim 2 wherein the air oxidizable excipient is Polysorbate 80 or Polysorbate 40 present in about 0.8×10⁻³ to 0.3 w/v %.

4. The suspension formulation of claim 3 wherein the air oxidizable excipient is Polysorbate 80 having peroxide value of 20 μequiv H₂O₂/mL or less.

5. The suspension formulation of claim 3 wherein the air oxidizable excipient is Polysorbate 80 having peroxide value of about 10-20 μequiv H₂O₂/mL.

6. The suspension formulation of claim 5 additionally comprising an effective amount of a pharmaceutically acceptable heavy metal chelator agent.

7. The suspension formulation of claim 6 wherein the heavy metal chelator agent is one, two or more heavy metal chelator agents selected from the group consisting of an acid or pharmaceutically acceptable salt of ethylenediamine-tetraacetate, ethyleneglycol-tetraacetate, diethylenetriamine-pentaacetate, hydroxyethylethylenediamine-triacetate, diaminocyclohexane-tetraacetate or 1,2-bis(2-aminophenoxy)ethane-N,N,N′,N′-tetraacetate.

8. The suspension formulation of claim 5 additionally comprising between about 0.01-0.3 w/v % of an edetate.

9. The suspension formulation of claim 8 additionally comprising an effective amount of pharmaceutically acceptable free radical inhibitor agent.

10. The suspension formulation of claim 9 wherein the free radical inhibitor agent is one, two, three or more antioxidants selected from the group consisting of Vitamin E, ascorbic acid, fumaric acid, malic acid, glutamic acid and tartaric acid.

11. The suspension formulation of any one of claims 5-10 additionally comprising 2.5-1,000 mL per gram pharmaceutically acceptable diluent wherein the diluent is a 10-150 mM buffered aqueous solution or provides for osmolality suitable for intramuscular injection to a human.

12. The formulation of claim 11 wherein the diluent is citrate or sodium phosphate buffer and wherein the formulation has initial pH of about pH 4 to 9.

13. An efficacy stabilized suspension formulation comprising an F1C, a pharmaceutically acceptable air oxidizable excipient, a pharmaceutically acceptable diluent and a pharmaceutically acceptable heavy metal chelator agent
wherein the F1C is androst-5-ene-3β,17β-diol or androst-5-ene-3β,7β,17β-triol;
wherein the diluent is a mixture of sodium phosphate mono basic and sodium phosphate dibasic in water;
wherein the air oxidizable excipient is Polysorbate 80 present in about 0.5% w/v;
wherein the heavy metal chelator agent is an edetate or pentetate present in about 0.01-0.05% w/v;
wherein the formulation has an initial pH of about 4-7.5 and an osmolality of about 229 to 343 mOsmol/kg or a initial pH and osmolality suitable for intramuscular injection.

14. The formulation of claim 13 wherein heavy metal chelator agent is an edetate.

15. A sterile suspension formulation in a closeable vessel prepared by the process of
(1) contacting androst-5-ene-3β,17β-diol, a diluent and at least one air oxidizable excipient to provide a suspension
or depleting oxygen dissolved in a suspension comprising water for injection, androst-5-ene-3β,17β-diol and at least one air oxidizable excipient
(2) replacing the headspace in the closeable vessel within which the suspension from step 1 resides;
(3) heating the vessel at a sterilization temperature of 121° C. for between about 15-45 min.

16. The sterile suspension formulation of claim 15 wherein one air oxidizable excipient is polysorbate 80.

17. The sterile formulation of claim 16 further comprising mannitol, benzalkonium chloride, sodium phosphate monobasic, sodium phosphate dibasic and an a heavy metal chelator agent wherein the chelator agent is an edetate or pentetate.

18. A method for treating an immune suppressive condition, a blood disorder deficiency or radiation exposure or a symptom thereof by administering to a subject having said condition or symptom a therapeutically effective amount of a suspension formulation comprising an F1C, a pharmaceutically acceptable aqueous-based diluent and at least one pharmaceutically acceptable, air oxidizable excipient wherein the F1C is androst-5-ene-3β,17β-diol, androst-5-ene-3β,7β,17β-triol or an ester or ether derivative of either of these compound and wherein the formulation
contains less than about 1-2 ppm of dissolved oxygen or is essentially free of dissolved oxygen
or contains less than about 25-160 ppm of lead equivalent of heavy metal, less than about 1-30 ppm or essentially free of heavy metal wherein the heavy metal is one, two, three or more metals selected from the group consisting of iron, cobalt, copper, chromium, vanadium
or has an initial peroxide value of 100 μequiv H₂O₂/mL or less.

19. The method of claim 18 wherein the condition or symptom thereof is associated with radiation exposure.

20. The method of claim 19 wherein the F1C is androst-5-ene-3β,17β-diol.

21. The method of claim 20 wherein the air oxidizable excipient is Polysorbate 80.

22. A method for treating an immune suppressive condition, a blood disorder deficiency or radiation exposure or a symptom thereof by administering to a human having said condition or symptom a therapeutically effective amount of a sterile suspension formulation prepared by the process of
(1) contacting androst-5-ene-3β,17β-diol, a diluent and at least one air oxidizable excipient to provide a suspension
or depleting oxygen dissolved in a suspension comprising water for injection, androst-5-ene-3β,17β-diol and at least one air oxidizable excipient
(2) replacing the headspace in the closeable vessel within which the suspension from step 1 resides;
(3) heating the vessel at a sterilization temperature of 121° C. for between about 15-45 min.

23. The method of claim 22 wherein the condition or symptom thereof is associated with radiation exposure.
